SEQUENCE LISTING (46 sequences)

SEQ ID NO: 1 - DNA, 20 bp, Artificial Sequence
  Synthetic Oligonucleotide
tgcaccaccaactgcttagc

SEQ ID NO: 2 - DNA, 20 bp, Artificial Sequence
  Synthetic Oligonucleotide
tcttctgggtggcagtgatg

SEQ ID NO: 3 - DNA, 24 bp, Artificial Sequence
  Synthetic Oligonucleotide
cagcagcgactctgaggaggaaca

SEQ ID NO: 4 - DNA, 24 bp, Artificial Sequence
  Synthetic Oligonucleotide
gcctccagcagaaggtgatccaga

SEQ ID NO: 5 - DNA, 24 bp, Artificial Sequence
  Synthetic Oligonucleotide
acctcgacacccgctaacaaatga

SEQ ID NO: 6 - DNA, 24 bp, Artificial Sequence
  Synthetic Oligonucleotide
aatgggcacgaaagatgagggaga

SEQ ID NO: 7 - DNA, 24 bp, Artificial Sequence
  Synthetic Oligonucleotide
tgtgtgtgctttgtggagggtact

SEQ ID NO: 8 - DNA, 24 bp, Artificial Sequence
  Synthetic Oligonucleotide
tgctggtctccaggtaacgaacaa

SEQ ID NO: 9 - DNA, 21 bp, Artificial Sequence
  Synthetic Oligonucleotide
ggtctgaccccaaacaccttc

SEQ ID NO: 10 - DNA, 20 bp, Artificial Sequence
  Synthetic Oligonucleotide
aacgggaaccaggacacatg

SEQ ID NO: 11 - DNA, 24 bp, Artificial Sequence
  Synthetic Oligonucleotide
cagtttgttggcggaagcgtgtaa

SEQ ID NO: 12 - DNA, 24 bp, Artificial Sequence
  Synthetic Oligonucleotide
aggatgtgcacaggctgtatcctt

SEQ ID NO: 13 - DNA, 21 bp, Artificial Sequence
  Synthetic Oligonucleotide
acaccaggtgaaccggccact

SEQ ID NO: 14 - DNA, 20 bp, Artificial Sequence
  Synthetic Oligonucleotide
ccttcggctcctcctgtgcc

SEQ ID NO: 15 - DNA, 24 bp, Artificial Sequence
  Synthetic Oligonucleotide
attgtcagacgacaaccaccacct

SEQ ID NO: 16 - DNA, 24 bp, Artificial Sequence
  Synthetic Oligonucleotide
ttccttcttcatggtcagtggcct

SEQ ID NO: 17 - DNA, 21 bp, Artificial Sequence
  Synthetic Oligonucleotide
ccttgtgcaatgtgaatgtgc

SEQ ID NO: 18 - DNA, 21 bp, Artificial Sequence
  Synthetic Oligonucleotide
cggagagtctcattttggcaa

SEQ ID NO: 19 - DNA, 24 bp, Artificial Sequence
  Synthetic Oligonucleotide
tgtgggaagccatcaggacgttca

SEQ ID NO: 20 - DNA, 24 bp, Artificial Sequence
  Synthetic Oligonucleotide
ccgcatgccgtacacgtagacatc

SEQ ID NO: 21 - DNA, 23 bp, Artificial Sequence
  Synthetic Oligonucleotide
gggatgacctgaagaaattgcta

SEQ ID NO: 22 - DNA, 25 bp, Artificial Sequence
  Synthetic Oligonucleotide
tgttgatatccaactctttgaacca

SEQ ID NO: 23 - DNA, 24 bp, Artificial Sequence
  Synthetic Oligonucleotide
aacgccaaacgcacgagtattacc

SEQ ID NO: 24 - DNA, 24 bp, Artificial Sequence
  Synthetic Oligonucleotide
tgaagttgttctcggcgaagctct

SEQ ID NO: 25 - DNA, 20 bp, Artificial Sequence
  Synthetic Oligonucleotide
tggcatctccacccgcagtc

SEQ ID NO: 26 - DNA, 24 bp, Artificial Sequence
  Synthetic Oligonucleotide
gagcttctccctgtaaatcgggcc

SEQ ID NO: 27 - DNA, 24 bp, Artificial Sequence
  Synthetic Oligonucleotide
gtgtggccgatgcagattactcgg

SEQ ID NO: 28 - DNA, 24 bp, Artificial Sequence
  Synthetic Oligonucleotide
ccactggacaggttgctgatgctg

SEQ ID NO: 29 - DNA, 24 bp, Artificial Sequence
  Synthetic Oligonucleotide
aggcttccatttgctgctgacaca

SEQ ID NO: 30 - DNA, 26 bp, Artificial Sequence
  Synthetic Oligonucleotide
tccatgccactctgacaaaaagtggg

SEQ ID NO: 31 - DNA, 24 bp, Artificial Sequence
  Synthetic Oligonucleotide
caaccagacgaggatggccaccta

SEQ ID NO: 32 - DNA, 24 bp, Artificial Sequence
  Synthetic Oligonucleotide
gactgctctggctgtcttggagga

SEQ ID NO: 33 - DNA, 24 bp, Artificial Sequence
  Synthetic Oligonucleotide
ggccgcaagacaggcttttcagat

SEQ ID NO: 34 - DNA, 24 bp, Artificial Sequence
  Synthetic Oligonucleotide
attgactccattgtcgccagcagc

SEQ ID NO: 35 - DNA, 24 bp, Artificial Sequence
  Synthetic Oligonucleotide
actgccccctgttgtcataggtcc

SEQ ID NO: 36 - DNA, 24 bp, Artificial Sequence
  Synthetic Oligonucleotide
acgtctgcttgagatgctcttgca

SEQ ID NO: 37 - DNA, 22 bp, Artificial Sequence
  Synthetic Oligonucleotide
gcgaagtctccccgaattagtg

SEQ ID NO: 38 - DNA, 24 bp, Artificial Sequence
  Synthetic Oligonucleotide
gtctctatggggagttaggttact

SEQ ID NO: 39 - DNA, 23 bp, Artificial Sequence
  Synthetic Oligonucleotide
cagcaactatgaaataatcgtag

SEQ ID NO: 40 - DNA, 24 bp, Artificial Sequence
  Synthetic Oligonucleotide
tccgcctccgcctcgacctccaac

SEQ ID NO: 41 - DNA, 22 bp, Artificial Sequence
  Synthetic Oligonucleotide
gactatctcccagcggcaggcc

SEQ ID NO: 42 - DNA, 23 bp, Artificial Sequence
  Synthetic Oligonucleotide
ccgggctccgagtcggtcagatc

SEQ ID NO: 43 - DNA, 28 bp, Artificial Sequence
  Synthetic Oligonucleotide
tcggactgaccacgttcagcggtgaagg

SEQ ID NO: 44 - DNA, 22 bp, Artificial Sequence
  Synthetic Oligonucleotide
aagccagccaatcaacgccgcg

SEQ ID NO: 45 - DNA, 21 bp, Artificial Sequence
  Synthetic Oligonucleotide
acagtccatgccatcactgcc

SEQ ID NO: 46 - DNA, 21 bp, Artificial Sequence
  Synthetic Oligonucleotide
gcctgcttcaccaccttcttg

(19) United States
(12) Patent Application Publication
(10) Pub. No.: US 2013/0136722 A1
(43) Pub. Date: May 30, 2013

(54) Methods of Ex Vivo Expansion of Blood Progenitor Cells, and Generation of Composite Grafts

(71) Applicant: The Board of Trustees of The University of Illinoi, Urbana, IL (US)

(72) Inventor: Nadim Mahmud, Chicago, IL (US)

(73) Assignee: The Board of Trustees of The University of Illinois, Urbana, IL (US)

(21) Appl. No.: 13/675,204

(22) Filed: Nov. 13, 2012

Related U.S. Application Data
(60) Provisional application No. 61/558,578, filed on Nov. 11, 2011.

(51) Int. Cl. A61K 35/28

(52) U.S. Cl. CPC A61K 35/28
USPC 424/93.7; 435/325; 435/6.12

(57) ABSTRACT

This invention provides methods and compositions of hematopoietic progenitor cells and hematopoietic stem cells, particularly methods for expanding populations of these cells types from biological sources.

### CROSS-REFERENCE TO RELATED APPLICATIONS

This application is related to U.S. provisional patent application Ser. No. 61/558,578, filed Nov. 11, 2011, the disclosure of which is incorporated by reference.

### FIELD OF THE INVENTION

This invention is related to methods and compositions of hematopoietic progenitor cells and hematopoietic stem cells, particularly methods for expanding populations of these cells types from biological sources wherein these cells are present.

### BACKGROUND OF THE INVENTION

Each year, 115,000 people in the United States develop leukemia or lymphoma and many of them die due to the lack of a curative therapy. Although there is no definitive way to prevent leukemia or lymphoma, it can be effectively treated using a variety of methodologies. However, many of these treatments also suppress or ablate endogenous hematopoietic stem cells (HSC) in the individual, leading to less aggressive use of chemotherapeutic drug or radiation dosing or length of treatment which reduces the effectiveness of the treatment with respect to long-term, disease free survival. These deficiencies can be addressed in the short term by exogenous hematopoietic stem cell transplantation. However, the difficulty in obtaining tissue-matched donors has limited widespread use of allogeneic HSC transplantation to treat these conditions. An alternative, human umbilical cord blood HSCs, are unsuitable for more than 90% of adult candidates due to an insufficient number of HSCs in cord blood isolates (see, Rocha & Gluckman, 2009, Br. J. Haematol. 147: 262-274). The limited number of HSCs in a single cord blood isolate is likely to account for the high rate of transplantation graft failure and delayed engraftment encountered frequently in adult recipients (Rocha & Gluckman, 2009, Id.; Laughlin et al., 2001, N. Engl. J. Med. 344: 1815-1822).

The hematopoietic stem cell (HSC) is the progenitor cell for all blood cells. Proliferation and differentiation of HSCs gives rise to the entire hematopoietic system. HSCs are believed to be capable of self-renewal—expanding their own population of stem cells—and they are pluripotent—capable of differentiating into any cell in the hematopoietic system. From this rare cell population, the entire mature hematopoietic system, comprising lymphocytes (B and T cells of the immune system) and myeloid cells (erythrocytes, megakaryocytes, granulocytes and macrophages) is formed. The lymphoid lineage, comprising B cells and T cells, provides for the production of antibodies, regulation of the cellular immune system, detection of foreign agents in the blood, detection of cells foreign to the host, and the like. The myeloid lineage, which includes monocytes, granulocytes, megakaryocytes as well as other cells, monitors for the presence of foreign bodies, provides protection against neoplastic cells, scavenges foreign materials, produces platelets, and the like. The erythroid lineage provides red blood cells, which act as oxygen carriers. As used herein, “stem cell” refers to hematopoietic stem cells and not stem cells of other cell types. Hematopoiesis is a complex process which involves a hierarchy of HSC and progenitor cells for each lineage from such cells, and can be influenced by a variety of external regulatory factors. To date attempts to create an in vitro environment which favors HSC self replication rather than commitment and differentiation has resulted in limited success. See, Jiang et al., 2002, Oncogene 21: 3295-3313; Guenahel et al., 2001, Exp. Hematol. 29: 1465-1473; Srour, 2000, Blood 96: 1609-1612; Berardi et al., 1995, Science 267: 104-108; and Heike et al., 2002, Biochim. Biophys. Acta 1592:313-321.

Blood cells are constantly replaced in the body by the process of hematopoiesis and damage to the hematopoietic system through disease or treatment of disease can cause particular deficiencies in different cell types. For example, myelosuppression and myeloablation are often seen as a result of cancer chemotherapy, due inter alia to the sensitivity of hematopoietic stem cells to chemotherapeutic agents. Bone marrow transplantation, either autologous or allogeneic, has been used to replace components of a functional hematopoietic system. Alternatively, purified stem cells may be reinfused into a patient to restore hematopoiesis in these compromised patients. It also has been found that administration of chemotherapeutic agents and/or cytokines mobilizes bone marrow stem cells into the peripheral blood such that peripheral blood can be harvested as a source of stem cells. In an autologous transplant setting it is often particularly desirable to purify stem cells from the bone marrow or peripheral blood to use as a graft as a way of purifying long-term repopulating cells free of contaminating tumor cells. However, these methods of autologous bone marrow repopulation have been hampered by the fact that tumor cells have been detected as high as 10% in mobilized peripheral blood collections and up to 80% in the mononuclear fraction from marrow.

Allogeneic stem cell sources include bone marrow donations and umbilical cord blood. However, in the case of bone marrow donation, like all tissue transplantation there is a need for histocompatibility antigen matches that limits the pool of potential donors and cannot be used for tissue “banking,” i.e., as a generic source of bone marrow-derived hematopoietic stem cells or progenitor cells. Umbilical cord blood is an alternative source of hematopoietic stem or progenitor cells but has limitations particularly for adult patients due to the fact that there are a limited number of HSC within a single cord blood (CB) unit; this likely accounts for the high rate of graft failure and delayed blood and immune cells reconstitution (engraftment) encountered with CB transplantation, particularly in adults (Laughlin et al., 2001, N. Engl. J. Med. 344: 1815-1822; Rocha & Gluckman, 2009, Br J Haematol. 147: 262-274).

Thus there remains a need in this art for methods relating to producing sufficient hematopoietic stem cells or progenitor cells or both for preventing or treating suppression or ablation of hematopoietic cells in patients having certain cancers or undergoing treatment for cancer.

### SUMMARY OF THE INVENTION

This application provides methods relating to producing from a biological source sufficient hematopoietic stem cells or progenitor cells or both for preventing or treating suppression or ablation of hematopoietic cells in patients having certain cancers or undergoing treatment for cancer, and compositions and pharmaceutical compositions of such cells. Also provided are methods of using said compositions and pharmaceutical compositions to prevent or treat hematopoietic cell suppression or ablation resulting from cancer chemotherapy or from bone marrow ablation incident to radiation or chemotherapeutic treatment for diseases such as leukemia or lymphoma.

In a first aspect, the invention provides methods for preparing an expanded population of hematopoietic progenitor cells from a biological source, comprising the step of culturing at least a portion of a hematopoietic progenitor cell preparation in a culture media containing valproic acid or other histone deacetylase inhibitor (HDACI) for a time and at a concentration wherein the population of hematopoietic progenitor cells is expanded. In particular embodiments, the biological source of the hematopoietic progenitor cells is bone marrow, peripheral blood and most particularly umbilical cord blood. In particular embodiments, said hematopoietic progenitor cells are grown in culture is grown for about 9 days after the addition of valproic acid, in which valproic acid is added at least twice (at 0 hour and 48 hours). In additional specific embodiments of the methods of the invention, the hematopoietic progenitor cells are grown in culture media containing valproic acid between about 7 and 10 days, particularly 9 days. Hematopoietic progenitor cells are advantageously cultured in media containing from about 0.5 mM to about 1.0 mM valproic acid, and particularly at a concentration of about 1 mM. Further advantageous embodiments of the invention comprise hematopoietic progenitor cells expanded in a culture media comprising at least one cytokine that is stem cell factor (SCF), Interleukin-1 (IL-1), Interleukin-2 (IL-2), Interleukin-3 (IL-3), Interleukin-4 (IL-4), Interleukin-5 (IL-5), Interleukin-6 (IL-6), Interleukin-7 (IL-7), Interleukin-8 (IL-8), Interleukin-9 (IL-9), Interleukin-10 (IL-10), Interleukin-11 (IL-11), Interleukin-12 (IL-12), erythropoietin (EPO), thrombopoietin (TPO), Granulocyte Colony-stimulating Growth Factor (G-CSF), Macrophage Colony-Stimulating Factor (M-CSF), Granulocyte-Macrophage Colony-Stimulating Factor (GM-CSF), Insulin-like Growth Factor-1 (IGF-1), Flt-3 ligand, or Leukemic Inhibitory Factor (LIF). In particularly advantageous embodiments, said media comprises Flt-3 ligand, TPO, IL-3 and SCF.

In another aspect, the invention provides expanded hematopoietic progenitor cell preparations produced according to the methods disclosed herein. Particularly for use in methods of treating or preventing suppression or ablation of hematopoietic stem cells in a cancer patient, the invention provides pharmaceutical compositions comprising a hematopoietic progenitor cell population as set forth herein and a pharmaceutically acceptable carrier or adjuvant, particularly embodiments thereof adapted for hematopoietic progenitor cells including in non-limiting example plasmalyte with 2.5% human serum albumin.

Also disclosed herein are methods for preventing or treating a cancer chemotherapy patient for hematopoietic sequellae of cancer chemotherapy, radiotherapy or any other biologic or physical agents resulting in myeloablation and bone marrow failure as a sequel, comprising the step of administering to the patient a pharmaceutical composition of hematopoietic progenitor cells prepared according to the methods set forth herein. In particularly advantageous embodiments, the hematopoietic sequella is neutropenia, thrombocytopenia or pancytopenia.

In another aspect, disclosed herein are methods for preparing composite cell preparations from umbilical cord blood comprising hematopoietic stem cells and hematopoietic progenitor cells, wherein said methods comprise the steps of culturing a portion of said umbilical cord blood cell preparation in the presence of valproic acid for a time and at a concentration wherein the population of hematopoietic progenitor cells is expanded, and combining said expanded portion with an unmanipulated portion of umbilical cord blood into the composite cell preparation. Alternative aspects provide methods for preparing composite cell preparations from umbilical cord blood comprising hematopoietic stem cells and hematopoietic progenitor cells, said methods comprising the steps of culturing a portion of said umbilical cord blood stem cell preparation in the presence of valproic acid for a time and at a concentration wherein the population of hematopoietic progenitor cells is expanded, and culturing a second portion of said umbilical cord blood stem cell preparation sequentially in the presence of 5-aza-2′-deoxycytidine and trichostatin A where in particular embodiments these agents are added to the cell culture media in a sequential fashion for a time and at a concentration wherein the population of hematopoietic stem cells is expanded, and combining said first and second expanded portions of hematopoietic stem cells and hematopoietic progenitor cells into the composite cell preparation. In particular embodiments, said hematopoietic progenitor cells are grown in culture for between about 16 to about 48 hours prior to the addition of valproic acid. In additional specific embodiments of the methods of the invention, the hematopoietic progenitor cells are grown in culture media containing valproic acid between about 7 and 10 days. Hematopoietic progenitor cells are advantageously cultured in media containing from about 0.5 mM to about 1.0 mM valproic acid, and particularly at a concentration of about 1 mM. In particular embodiments, said hematopoietic stem cells are grown in culture is grown for between about 5 to about 7 days following the sequential addition of 5-aza-2′-deoxycytidine and trichostatin A, where in particular embodiments 5-aza-2′-deoxycytidine is added to the culture 16 hr after the cell culture was initiated and then trichostatin A added at 48 hr with a change in media. In additional specific embodiments of the methods of the invention, the hematopoietic stem cells are grown in culture media containing 5-aza-2′-deoxycytidine and trichostatin A according to sequential administration of 5-aza-2′-deoxycytidine and trichostatin A over the first two days of culture and then grown for an additional 7 days before harvesting. Hematopoietic progenitor cells are advantageously cultured in media containing from about 0.5 μM to about 1.0 μM 5-aza-2′-deoxycytidine and from about 2.5 ng/ml to about 5 ng/ml trichostatin A, and particularly at a concentration of about 1.0 μM 5-aza-2′-deoxycytidine and about 5 ng/ml trichostatin A. Further advantageous embodiments of the invention comprise methods for expanding hematopoietic progenitor cells or hematopoietic stem cells or both in a culture media comprising at least one cytokine that is stem cell factor (SCF), Interleukin-1 (IL-1), Interleukin-2 (IL-2), Interleukin-3 (IL-3), Interleukin-4 (IL-4), Interleukin-5 (IL-5), Interleukin-6 (IL-6), Interleukin-7 (IL-7), Interleukin-8 (IL-8), Interleukin-9 (IL-9), Interleukin-10 (IL-10), Interleukin-11 (IL-11), Interleukin-12 (IL-12), erythropoietin (EPO), thrombopoietin (TPO), Granulocyte Colony-stimulating Growth Factor (G-CSF), Macrophage Colony-Stimulating Factor (M-CSF), Granulocyte-Macrophage Colony-Stimulating Factor (GM-CSF), Insulin-like Growth Factor-1 (IGF-1), Flt-3 ligand, or Leukemic Inhibitory Factor (LIF). In particularly advantageous embodiments, said media comprises Flt-3 ligand, TPO, IL-3 and SCF.

In another aspect, the invention provides composite cell preparations comprising expanded hematopoietic progenitor cell preparations produced according to the methods disclosed herein in combination with unmanipulated umbilical cord blood cells or an expanded population of hematopoietic stem cells. Useful embodiments of said composite cell preparation are provided as a pharmaceutical composition thereof further comprising a pharmaceutically acceptable carrier or adjuvant, particularly embodiments thereof adapted for hematopoietic stem cells and hematopoietic progenitor cells including in non-limiting example plasmalyte with 2.5% human serum albumin.

Also disclosed herein are methods for repopulating bone marrow in an animal with hematopoietic stem cells or hematopoietic progenitor cells or both, comprising the step of administering to an animal in need thereof a pharmaceutical composition of a composite composition of expanded hematopoietic progenitor cell preparations produced according to the methods disclosed herein in combination with unmanipulated umbilical cord blood cells or an expanded population of hematopoietic stem cells. In particular embodiments, said first portion of expanded hematopoietic progenitor cells comprises about one third of the composition and said second portion of unmanipulated umbilical cord blood cells or in the alternative an expanded population of hematopoietic stem cells comprises about two thirds of the composition.

In yet another aspect, provided herein are methods for assessing a cell preparation comprising hematopoietic progenitor cells and hematopoietic stem cells for the capacity for treating or preventing hematopoietic sequellae of cancer chemotherapy, the method comprising the steps of assaying said cell population for expression of Alox5, F2RL2, S100A8, Cyp11A1 or Collagen14A1 genes or the gene products thereof, wherein detecting expression of Alox5 and increased expression of F2RL2, S100A8, Cyp11A1 or Collagen14A1 in said cell population that is greater than said gene expression in unmanipulated cell preparations indicates that the cell preparation has a capacity for treating or preventing hematopoietic sequellae of cancer chemotherapy, radiation therapy or exposure to high dose radiation comprising complete ablation of bone marrow blood producing capacity.

In a further aspect, provided herein are methods for assessing a cell preparation comprising hematopoietic progenitor cells (that provides short term progenitor cells that maintain the hematopoietic cell system in a patient as a bridge prior to repopulation of bone marrow by long term hematopoietic stem cells) and hematopoietic stem cells for a capacity to repopulate hematopoietic stem cells in bone marrow in a patient in need thereof, the method comprising the steps of assaying said cell population for expression of Alox5, F2RL2, S100A8, Cyp11A1 or Collagen14A1 genes or the gene products thereof, wherein detecting expression of Alox5 and expression of F2RL2, S100A8, Cyp11A1 or Collagen14A1 in said cell population that is greater than said gene expression in unmanipulated cell preparations indicates that the cell preparation has a capacity to repopulate long term hematopoietic stem cells in bone marrow for a sustained period of time.

It is an advantage of the methods and compositions set forth herein to provide expanded populations of hematopoietic progenitor cells or hematopoietic stem cells, or both, that have been expanded by growth in culture to achieve clinically useful numbers of hematopoietic progenitor cells or hematopoietic stem cells or both. Moreover, it is a further advantage of the methods and compositions set forth herein to provide expanded populations of hematopoietic progenitor cells or hematopoietic stem cells or both that retain the properties and characteristics of hematopoietic progenitor cells or hematopoietic stem cells or both, wherein said hematopoietic progenitor cells can provide hematopoietic cells or said hematopoietic stem cells or both to an individual in need thereof. Yet a further advantage of the methods and compositions disclosed herein is that they provide the ability to address clinical deficits experienced by patients with cancers like leukemia or undergoing chemotherapy related to hematopoiesis, for example blood cell deficiencies such as leukocytopenia or neutropenia resulting from hematopoietic progenitor cell or stem cell suppression or ablation, or by patients whose bone marrow has been ablated as part of or as a consequence of treatment of leukemia or lymphoma. In certain advantageous embodiments, the methods and compositions provided herein provide a “short-term” hematopoietic progenitor cell population that can be to be used in acute situations such as preventing or treating leukocytopenia or neutropenia resulting from cancer chemotherapy. In certain other advantageous embodiments, the methods and compositions provided herein provide composite compositions comprising a “short-term” hematopoietic progenitor cell population in combination with hematopoietic stem cell preparations, that permit maintenance of hematopoiesis in a patient in need thereof while the stem cells in said composite composition repopulate a patient's bone marrow. As set forth herein, VPA treatment alone showed the highest expansion of primitive CD34⁺CD90⁺ cells and progenitor cells. Transplantation of VPA-expanded short-term progenitor cells along with unmanipulated CB graft can improve patient survival by bridging the period of low blood cell count (a.k.a., the neutropenic period) thereby preventing infection following CB transplantation and ensuring sustained blood cell production from the unmanipulated CB graft. Similar advantages accrue in embodiments comprising expanded hematopoietic stem cell cultures treated ex vivo with 5-aza-deoxycytidine and TSA.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1A shows the results of fluorescence-activated cell sorting (FACS) analyses on the effects of chromatin modifying agents (CMAs) on expansion of cord blood (CB)-derived, CD34⁺CD90⁺ cells following 9 days of culture, wherein the cells were treated with the chromatin modifying agents set forth at the top of each scatter plot. Flow cytometric analysis of CB stem/progenitor cell phenotype was determined using mAb for CD34 and CD90 or their matched isotype control. The flow cytometry profiles of NA, 5azaD/NA and SAHA treated cultures are representative of two experiments while all other CMAs tested are representative of three independent experiments.

FIG. 1B shows the fold expansion of total nucleated cells (TNC), CD34⁺ cells and CD34⁺CD90⁺ cells following 9 days of culture in the presence of the CMA indicated, wherein the fold expansion was determined by dividing the total number of viable cells at the end of 9 days of culture expressing the indicated phenotype by the number of viable input cells expressing the same phenotype. The fold expansion of TNC, CD34⁺ cells and CD34⁺CD90⁺ cells was determined by dividing the total number of viable cells at the end of culture expressing the phenotype by the input number of viable cells expressing the same phenotype. * This bar graph represents the mean±SE of 3 independent experiments. ** This bar graph represents the mean of 2 independent experiments.

FIG. 2A shows the functional potential of CMA expanded CB cells as fold expansion of colony-forming cells (CFC) from their initial number following ex vivo culture of CD34⁺ cells. Data expressed as mean±SEM of three independent experiments.

FIG. 2B shows the functional potential of CMA expanded CB cells as fold expansion of cobblestone area-forming cells (CAFC) from their initial number following ex vivo culture of CD34⁺ cells. Data expressed as mean±SEM of three independent experiments.

FIG. 2C shows the in vivo hematopoietic reconstitution capacity of ex vivo expanded CB cells cultured with various CMAs and transplanted in NOD/SCID mice. 1×10⁴ primary CD34⁺CD90⁺ cells or an equal initial number of the expanded (5azaD/TSA, VPA, or control) culture product of CD34⁺CD90⁺ cells were transplanted in each mouse intravenously using the tail vein. The percentage of human CD45⁺ cells indicate the percent of human hematopoietic cells present in mouse bone marrow after transplantation as determined by flow cytometry.

FIG. 2D shows representative flow cytometric analyses demonstrating the in vivo multi-lineage differentiation capacity of expanded CB cells cultured with various CMAs to both myeloid and lymphoid lineages following transplantation. The in vivo multilineage differentiation capacity of expanded CB cells to both myeloid and lymphoid lineages following transplantation is shown in representative flow cytometric analysis. Unfractionated mouse BM cells following harvest were stained for human CD45, CD19, CD33, CD34 and CD41 to assess multilineage human hematopoietic engraftment.

FIG. 2E shows the in vivo bone marrow homing capacity of CD34⁺ cells. The frequency of SRC present in the primary CD34⁺CD90⁺ cells prior to (day 0) and following culture (day 9) in the presence or absence of chromatin modifying agents was determined by limiting dilution approach. NOD/SCID mice were transplanted with increasing doses (1000, 2000, 5000, 10000, 20000, 50000, 100000) of CD34⁺CD90⁺ cells calculated to be present in the purified primary CB CD34⁺ cell fraction or the cellular products of cytokines alone or 5azaD/TSA- or VPA-expanded cultures initiated with equal input numbers of CD34⁺CD90⁺ cells.

FIG. 2F shows the homing efficiency of cord blood cells expanded in control (cytokine-only treated) cultures was 0.05% while 5azaD/TSA- and VPA-expanded CB cells possessed 0.39% and 1.68% homing efficiency, respectively (control vs. 5azaD/TSA, P=0.002; 5azaD/TSA vs. VPA, P=0.008).

FIG. 3A shows the effect of chromatin modifying agents on the expression of genes implicated in HSC self-renewal and differentiation as determined by real time quantitative PCR in primary CD34⁺ cells (day 0) or CD34⁺ cells re-isolated from expansion culture at day 3 and day 9.

FIG. 3B shows the effect of 5azaD/TSA or VPA on the expression of genes implicated in HSC self-renewal and differentiation as determined by real time quantitative PCR in CD34⁺ cells re-isolated from expansion culture at day 3. The transcript levels of genes were also studied in re-isolated CD34⁺ cells following expansion culture in the presence of 5azaD/TSA, VPA, or control (Ezh2: Control vs. 5azaD/TSA p<0.005, Control vs. VPA p<0.05; GATA1: Control vs. 5azaD/TSA p<0.001 Control vs. VPAp <0.001; HoxB4: Control vs. 5azaD/TSAp <0.005, Control vs. VPA p<0.05; Bmi1: Control vs. VPA p<0.005). All data presented here represents mean±SE of three independent experiments except for the transcript values at day 3.

FIG. 4A shows the intersection of global gene expression arrays from CB CD34⁺ cells expanded in various CMAs used to identify genes linked with HSC expansion or HSC maintenance. Common genes were identified based on their differential expression on expanded CD34⁺ cells from 5azaD/TSA, VPA and control cultures linked with their distinct in vivo HSC repopulation function were analyzed using global microarray as described below. Using this strategy HSC expansion and HSC maintenance gene lists were generated from differentially expressed genes between these three pairs of expanded CD34⁺ cell populations (5azaD/TSA vs. VPA, 5azaD/TSA vs. Control, and VPA vs. Control). 113 HSC expansion genes were identified which are differentially expressed between 5azaD/TSA vs. Control and 5azaD/TSA vs. VPA. Similarly 278 HSC maintenance genes were identified which are differentially expressed between 5azaD/TSA vs. Control and VPA vs. Control.

FIG. 4B shows a heat map generated by analyzing differential gene expression of primary or ex vivo expanded CD34⁺ cells based on their in vivo hematopoietic reconstitution function, and indicates that 88 genes passed with an r-value of 0.85, demonstrating a high level of correlation between the expression pattern of these genes and the regenerative capacity of the samples. Cluster analysis of global gene expression is displayed from CMA-expanded CD34⁺ cells. CD34⁺ cells possessing regenerative capacity (D0 primary, and D9 5azaD/TSA- or D9 VPA-expanded) were assigned a regeneration capacity grade of 2 for hematopoietic reconstitution, while samples lacking in vivo hematopoietic reconstitution capacity function (non-regenerative samples: D9 control and CMA added in reversed sequence as TSA/5azaD D9) were assigned a grade of 0.01.

FIG. 4C shows that Principal Component Analysis of the 88 genes reveals that samples with regenerative capacity are clustered together, while samples without regenerative capacity are clustered separately in a distinct region indicating their possible unique gene function using primary or ex vivo expanded CD34⁺ cells with and without hematopoietic regenerative capacity.

FIG. 4D shows the results of Global functional analysis using Ingenuity Pathway Analysis revealed top signaling networks within 88 differentially expressed genes based on presence or absence of in vivo hematopoietic regeneration capacity of CMA expanded CB CD34⁺ cells.

FIG. 4E shows the levels of an inflammatory mediator (LTB4) in conditioned media following expansion of CD34⁺ cells with or without CMA for 9 days shown as mean±SE of triplicate wells. The concentration of LTB4 in conditioned media at day 9 of expansion culture was measured by an AChE competitive ELISA.

FIG. 5A shows real time quantitative PCR validation of genes identified in global gene expression microarray analysis from genes linked with the in vivo HSC expansion function of CMA expanded CD34⁺ cells shown as mean±SE of 3 independent experiments.

FIG. 5B shows real time quantitative PCR validation of genes identified in global gene expression microarray analysis from genes linked with both the in vivo HSC maintenance and expansion function of CMA expanded CD34⁺ cells (except Alox5 which is differentially expressed in the HSC maintenance gene set) shown as mean±SE of 3 independent experiments.

FIG. 5C shows the methylation levels of promoter CpG sites of several genes which are differentially expressed between HSC expansion and HSC maintenance groups as analyzed by gyro-sequencing. Primary (day 0) or CD34⁺ cells expanded in 5azaD/TSA (day 3) or VPA (day 3) were utilized to obtain genomic DNA which was bisulfite treated and used to measure methylation levels in CpG sites near the promoter area of each gene.

FIG. 6A shows changes in DNA methylation levels (LINE-1) of samples derived from primary or ex vivo expanded CD34⁺ cells expanded in 5azaD/TSA, VPA or control cultures. LINE-1 assay show global methylation levels in repetitive DNA elements using genomic DNA from enriched CD34⁺ cells. These results indicate that 5azaD/TSA treatment was capable of resulting in significant but transient DNA hypomethylation of CD34⁺ cells in contrast to uncultured or expanded CD34⁺ cells in VPA or control cultures. In the absence of 5azaD/TSA treatment during culture the methylation levels of CpG sites remained elevated.

FIG. 6B shows changes in histone acetylation levels of samples derived from primary or ex vivo expanded CD34⁺ cells as demonstrated by Chromatin immunoprecipitation (ChiP) assays showing histone H4 acetylation levels in chromatin prepared from CD34⁺ cells expanded in 5azaD/TSA, VPA or control cultures. Chromatin was immunoprecipitated using anti-acetylated histone H4 antibody and chromatin bound DNA was PCR amplified using promoter specific primers. GAPDH was used as an internal control. No antibody and matched isotype controls were included as negative controls, and input chromatin was included as a positive control. Increased histone H4 acetylation of the promoter regions of HoxB4, Bmi1, and GATA2 genes corresponded with their higher transcript levels in both 5azaD/TSA and VPA expanded CD34⁺ cells in contrast to control cultures. More promoter fragments of Bmi1, HoxB4, and GATA2, and fewer fragments of Pu.1 were amplified in 5azaD/TSA as compared to control and VPA expanded CD34⁺ cells. Notably, VPA expanded CD34⁺ cells showed intermediate levels of histone H4 acetylation for Bmi1 and HoxB4 genes as compared to control cultures. Together, these data support that 5azaD/TSA- and VPA-expanded CD34⁺ cells increases histone H4 acetylation of the promoter sites of genes whose transcription level correlated with their degree of acetylation.

### DETAILED DESCRIPTION OF PREFERRED EMBODIMENTS

This application provides methods relating to producing from a biological source sufficient hematopoietic stem cells or progenitor cells or both for preventing or treating suppression or ablation of hematopoietic cells in patients having certain cancers or undergoing treatment for cancer, and compositions and pharmaceutical compositions of such cells. Also provided are methods of using said compositions and pharmaceutical compositions to prevent or treat hematopoietic cell suppression or ablation resulting from cancer chemotherapy or from bone marrow ablation incident to radiation or chemotherapeutic treatment for diseases such as leukemia or lymphoma.

Administration of hematopoietic progenitor cells (HPC) or hematopoietic stem cells (HSC) to patients undergoing cancer chemotherapy or after radiation or chemotherapeutic agent therapy for leukemia or lymphoma has been unsatisfactory in most clinical situations due to the difficulty in producing sufficient hematopoietic progenitor cells or hematopoietic stem cells from biological sources such as umbilical cord blood (CB). This limitation is overcome if the number of transplantable HSC within a single CB unit were expanded. There have been attempts in the art to use molecules including notch ligand, prostaglandin (PGE2), pleiotrophin, and aryl hydrocarbon receptor antagonist as positive stimulators of HSC although none of these molecules by itself showed potency (Delaney et al., 2010, Nat. Med. 16:232-236; Boitano et al., 2010, Science 329: 1345-1348; Himburg et al., 2010, Nat. Med. 16: 475-482; Goessling et al., 2011, Cell Stem Cell 8: 445-458). Current ex vivo expansion strategies in clinical trials using notch ligand or marrow stromal cell co-culture primarily expand short-term progenitors and rely on a second unmanipulated CB graft for long-term blood cell production (Himburg et al., 2010, Id.; Goessling et al., 2011, Id.). The ex vivo manipulation of HSC has significant therapeutic implications, including the use of expanded CB grafts for transplantation as curative therapy for both malignant and non-malignant blood disorders.

Methods of bone marrow expansion have been developed, however, expansion of stem cells is not as straightforward as expansion from a mature population. First, stem cells are very rare and, therefore, the number of stem cells isolated from any source will be very small. This reduces the size of the population that can be used to initiate the culture system. Second, the goal in stem cell expansion is not just to produce large quantities of mature cells, but also to retain stem cells and to produce many immature progenitor cells, which are capable of rapidly proliferating and replenishing mature cell types depleted in the patient. Upon reinfusion into a patient, the mature cells are cleared quickly whereas stem cells home to the marrow where long-term engraftment can occur (engraftment assays may be measured using for example, SCID mice using techniques well known to those of skill in the art). In addition, immature progenitor cells can produce more cell types and more numbers of cells than mature cells, thus providing short-term hematopoietic recovery.

Unlike whole bone marrow, stem cell replacement does not restore mature hematopoietic cells immediately. Due to the time necessary to generate mature cells from re-infused stem cells, there is a lag during which the patient remains immunocompromised. Thus, there is an advantage to expand purified (tumor-free) stem cells ex vivo to generate a cell population having a greater number of stem cells, and a further advantage in preparing such a cell population comprising slightly more differentiated hematopoietic cells, to provide both short- and long-term hematopoietic recovery.

Despite the availability of numerous methods for in vitro expansion of HSCs in culture, these methods remain inadequate for the production of HSCs for transplantation that maintain self-renewal capacity and multipotency. Although the molecular signature that defines an HSC has recently been described, the patterns of gene expression that lead to HSC self replication rather than commitment remain unknown (Santos et al., 2002, Science 298:597-600; Ivanova et al., 2002, Science, 298:601-604).

The invention provides methods for producing cultures of hematopoietic stem cells or hematopoietic progenitor cells or both, expanded by ex vivo culture from a biological source such as umbilical cord blood which comprises such cells of obtaining compositions having such cells in amounts useful for clinical administration to patients in need thereof. Compositions enriched for hematopoietic stem cells, hematopoietic progenitor cells or both are also provided by the invention. It is contemplated that the HSCs and the enriched populations of cells obtained therefrom may be used in all therapeutic uses for which HSC are presently employed, including in non-limiting example bone marrow transplants and administration to cancer chemotherapy patients to prevent or treat leucopenia, neutropenia, thrombocytopenia or other deficits in the hematopoietic cell compartment of the blood by cancer chemotherapy. Therapeutic methods using HSCs are well known to those of skill in the art (e.g., see U.S. Pat. No. 6,368,636).

Compositions for Expanding Hematopoietic Stem or Progenitor Cells In Vitro

The present invention employs compositions that prevent HSC methylation and acetylation which occurs as parts of normal cellular differentiation in culture in order to retain HSC multipotency by mitigating gene silencing, which are involved in self renewal of primitive hematopoietic stem cells capable of long term sustained blood cell production following transplantation in a host (human). Prevention of gene silencing for genes that disrupt symmetrical or asymmetrical self renewing cell division for expansion or maintenance of HSC during in vitro expansion culture is desirable in the practice of the methods set forth herein. The present section provides a brief discussion of DNA methylation inhibitors and histone deacetylase inhibitors that may be used for the methods described herein. The methods and compositions described in the patents recited below for producing and identifying inhibitors of histone deacetylase (HDACI) and inhibitors of DNA methylation (IDM) may be adapted to identify additional compounds that will be useful in the present invention.

In the culture conditions of the present invention, which may employ various cytokine combinations, varying amounts of IDMs and HDACIs may be added. The IDM and HDACI may be added concurrently at the beginning of the cell culture. Alternatively either the IDM or the HDACI agent may be added first and the other agent may be added later. For example, in preferred embodiments, the HDACI was added 48 hours after the initial exposure of the cells to IDM, a strategy which resulted in a 10.5 to 12-fold increase in CD34⁺CD90⁺ cells and 7-fold increase in in vivo repopulating transplantable hematopoietic stem cells. Those of skill in the art will be able to modify the time of exposure to the two types of agent, the amount of agent to be added and the order in which the agents are added to optimize HSC expansion. Of course, it should be understood that use of either the IDM or the HDACI alone in the HSC expansion culture also may be beneficial in producing an increase in HSC expansion and improving the reprogramming of hematopoietic cells.

a. Inhibitors of DNA Methylation (IDM)

DNA methylation is a postreplicative covalent modification of DNA that is catalyzed by the DNA methyltransferase enzyme (DNMT) (Koomar et al., 1994, Nucl. Acids Res. 22:1-10; and Bestor et al., 1988, J. Mol. Biol. 203:971-983). In vertebrates, the cytosine moiety at a fraction of the CpG sequences is methylated (60-80%) in a nonrandom manner generating a pattern of methylation that is gene and tissue specific (Yisraeli and M. Szyf, 1985, In DNA methylation: Biochemistry and Biological significance, pp. 353-378, Razin et al., (Ed), Springer-Verlag, N.Y.). It is generally believed that methylation in regulatory regions of a gene is correlated with a repressed state of the gene (Yisraeli and Szyf, 1985, In DNA methylation: Biochemistry and Biological significance, pp. 353-378, Razin et al., (Ed), Springer-Verlag, N.Y.; and Razin et al., 1991, Microbiol. Rev. 55:451-458). DNA methylation can repress gene expression directly, by inhibiting binding of transcription factors to regulatory sequences or indirectly, by signaling the binding of methylated-DNA binding factors that direct repression of gene activity (Razin et al., 1991, Microbiol. Rev. 55:451-458).

It is well established that regulated changes in the pattern of DNA methylation occur during development and cellular differentiation (Razin et al., 1991, Microbiol. Rev. 55:451-458; and Brandeis et al., 1993, Bioessays 13:709-713). The pattern of methylation is maintained by the DNA MeTase at the time of replication and the level of DNMT activity and gene expression is regulated with the growth state of different primary (Szyf et al., 1985, J. Biol. Chem. 260:8653-8656) and immortal cell lines (Szyf et al., 1991, J. Bol. Chem. 266:10027-10030). This regulated expression of DNMT has been suggested to be critical for preserving the pattern of methylation. It is the inhibition of such DNA methylation in cultures of HSC that is useful in the methods of the present invention.

Methods and compositions for inhibiting DNA methylation are well known to those of skill in the art. Such methods are disclosed in for example U.S. Pat. No. 6,184,211, which is incorporated herein by reference in its entirety and describes a reduction of the level of DNA methylation through inhibitors and antagonists in order to inhibit the excessive activity or hypermethylation of DNMT in cancer cells to induce the original cellular tumor suppressing program, to turn on alternative gene expression programs, to provide therapeutics directed at a nodal point of regulation of genetic information, and to modulate the general level of methylase and demethylase enzymatic activity of a cell to permit specific changes in the methylation pattern of a cell. Such methods and compositions may be used in the present invention to promote expansion of HSC in culture.

U.S. Pat. No. 6,255,293 is specifically incorporated herein by reference as providing a teaching of demethylation of cells using methods and compositions relating to demethylating agents such as 5-aza-2′-deoxycytidine. Use of such a compound as the demethylation compound is particularly useful in the present invention as protocols of 5-aza-2′-deoxycytidine treatment of patients were approved in the past and further used in the U.S. for other purposes, such as for use as an anticancer drug which induces cellular differentiation and enhanced expression of genes involved in tumor suppression, immunogenicity and programmed cell death. Thus, the use of 5-aza-2′-deoxycytidine and derivatives and analogs thereof in the HSC expansion methods of the present invention is specifically contemplated. It has been recognized that administration of this compound blocks DNA methylation. See, for example, Thibault et al, (1998), Momparler et al, (1997), Schwartsmann et al., (1997), Willemze et al, (1997) and Momparler, (1997), Reik et al., (2001), Blau, (1992), Jones et al., 2001.

Other agents for causing demethylation of methylated DNA or for preventing methylation of DNA also may be used in addition to or in combination with 5-aza-2′-deoxycytidine include but are not limited to 5,6-dihydro-5-azacytidine, 5-azacytidine, and 1-beta-D-arabinofuranosyl-5-azacytidine. See Antonsson et al. (1987), Covey et al. (1986), and Kees et al. (1995). Any compound known to be a cytosine specific DNA methyltransferase inhibitor would be expected to be operable in the present invention. Any such compound can be readily tested without undue experimentation in order to determine whether or not it works in the context of the present invention in the same manner as 5-aza-2′-deoxycytidine, for example by repeating the experiments of the present examples with each proposed demethylating agent.

b. Histone Deacetylase Inhibitors (HDACI)

Histone deacetylase and histone acetyltransferase together control the net level of acetylation of histones. Inhibition of the action of histone deacetylase results in the accumulation of hyperacetylated histones, which in turn is implicated in a variety of cellular responses, including altered gene expression, cell differentiation and cell-cycle arrest. Recently, trichostatin A and trapoxin A have been reported as reversible and irreversible inhibitors, respectively, of mammalian histone deacetylase (see e.g., Yoshida et al, 1995, Bioassays, 17(5): 423-430). Trichostatin A has also been reported to inhibit partially purified yeast histone deacetylase (Sanchez del Pino et al, 1994 Biochem. J., 303: 723-729).

In the present invention, trichostatin A is used as an HDACI. Trichostatin A is an antifungal antibiotic and has been shown to have anti-trichomonal activity as well as cell differentiating activity in murine erythroleukemia cells, and the ability to induce phenotypic reversion in sis-transformed fibroblast cells (see e.g. U.S. Pat. No. 4,218,478; Yoshida et al., 1995, Bioassays, 17: 423-430 and references cited therein). Alternatively, Trapoxin A, a cyclic tetrapeptide, which induces morphological reversion of v-sis-transformed NIH3T3 cells may be used in the present invention as the HDACI (Yoshida and Sugita, 1992, Jap. J. Cancer Res., 83: 324-328).

Other HDACI compounds are well known to those of skill in the art. For example, U.S. Pat. No. 6,068,987, specifically incorporated herein by reference describes a number of cyclic tetrapeptides structurally related to trapoxin A as inhibitors of histone deacetylase. Depsipeptide is another agent that has commonly been used as an HDACI (Ghoshal et al., 2002, Mol. Cell. Biol., 22: 8302-19). U.S. Pat. No. 6,399,568, incorporated herein by reference in its entirety, describes additional cyclic tetrapeptide derivatives that may be used as useful HDACI compounds in the HSC expansion methods of the present invention.

Methods of HSC Expansion

The present invention provides methods of expanding a population of cells substantially enriched in hematopoietic progenitor cells or hematopoietic stem cells by culturing the cells in the presence of valproic acid or a combination of 5-aza-deoxycytidine or TSA, respectively. The hematopoietic progenitor or stem cells used in the expansion method are advantageously substantially free of stromal cells. The method may be performed in closed, perfusable, culture containers or may be performed in an open culture system. A “closed culture” is one which allows for the necessary cell distribution, introduction of nutrients and oxygen, removal of waste metabolic products, optional recycling of hematopoietic cells and harvesting of hematopoietic cells without exposing the culture to the external environment, and does not require manual feeding or manual manipulation before the cells are harvested.

As used herein, “stem cells” refers to animal, especially mammalian, preferably human, hematopoietic stem cells and not stem cells of other cell types. “Stem cells” also refers to a population of hematopoietic cells having all of the long-term engrafting potential in vivo. Animal models for long-term engrafting potential of candidate human hematopoietic stem cell populations include the SCID-hu bone model (Kyoizumi et al., 1992, Blood 79: 1704-1711; Murray et al., 1995, Blood 85: 368-378) and the in utero sheep model (Zanjani et al., 1992, J. Clin. Invest. 89: 1179); for a review of animal models of human hematopoiesis, see Srour et al., 1992, J. Hematother. 1: 143-153 and the references cited therein. At present, in vitro measurement of stem cells is achieved through the long-term culture-initiating cell (LTCIC) assay, which is based on a limiting dilution analysis of the number of clonogenic cells produced in a stromal co-culture after 5-8 weeks (Sutherland et al., 1990, Proc. Nat'l Acad. Sci. 87: 3584-3588). The LTCIC assay has been shown to correlate with another commonly used stem cell assay, the cobblestone area forming cell (CAFC) assay, and with long-term engrafting potential in vivo (Breems et al., 1994, Leukemia 8: 1095).

The cell population used in the present invention is preferably an enriched cell population, in order to maximize the content of stem and early progenitor cells in the expanded cell population. An example of an enriched stem cell population is a population of cells selected by expression of the CD34 marker. In LTCIC assays, a population enriched in CD34⁺ cells generally has an LTCIC frequency in the range of 1/50 to 1/500, more usually in the range of 1/50 to 1/200. Preferably, the cell population will be more highly enriched for stem cells than that provided by a population selected on the basis of CD34⁺ expression alone. By use of various techniques described more fully below, a highly enriched stem or progenitor cell population may be obtained. A highly enriched stem cell population will typically have an LTCIC frequency in the range of ⅕ to 1/100, more usually in the range of 1/10 to 1/50. Preferably it will have an LTCIC frequency of at least 1/50. Exemplary of a highly enriched stem cell population is a population having the CD34⁺/Thy-1⁺/LIN⁻ phenotype as described in U.S. Pat. No. 5,061,620. A population of this phenotype will typically have an average LTCIC frequency of approximately 1/20 (Murray et al., 1995 supra; Lansdorp et al., 1993, J. Exp. Med. 177: 1331). It will be appreciated by those of skill in the art that the enrichment provided in any stem cell population will be dependent both on the selection criteria used as well as the purity achieved by the given selection techniques. Valproic acid is useful, as a particular embodiment of HDACI, for expanding short term hematopoietic progenitor cells present in human umbilical cord blood as described previously (Majeti et al., 2007, Cell Stem Cell 1: 635-645).

As used herein, the term “expanded” or “expansion” is intended to mean an increase in cell number from the hematopoietic progenitor cells or hematopoietic stem cells or both used to initiate the culture. “Substantially free of stromal cells” shall mean a cell population which, when placed in a culture system as described herein, does not form an adherent cell layer.

As used herein, “composite cell preparation” is a cell population, particularly a cell population expanded in vitro comprising an expanded hematopoietic progenitor cell population, particularly one expanded in the presence of an HDAC1 molecule and, alternatively, an unmanipulated hematopoietic stem cell population or an expanded stem cell population that was expanded in the presence of IDM molecule or an HDAC1 molecule.

As used herein, “hematopoietic progenitor cell” means a cell or cell population that can produce by hematopoietic differentiation particular cells in the hematopoietic lineage. In particular embodiments, such cells comprise a phenotype of cell surface markers (or the absence thereof) that include Lin⁻CD34⁺CD38⁻CD45RA⁻CD90⁻.

As used herein, an “unmanipulated” hematopoietic progenitor cell or stem cell population, for example, from umbilical cord blood comprises an uncultured umbilical cord blood graft or initial biological material (cells) derived from umbilical cord blood used to initiate expansion culture but without treatment with HDACI, IDM compounds or cytokines.

As used herein, “short-term” hematopoietic cell grown means that the hematopoietic cells, particularly hematopoietic progenitor cells, can sustain blood cell production for weeks to months.

As used herein, “long-term” hematopoietic cell grown means that the hematopoietic cells, particularly hematopoietic stem cells, can sustain life long blood cell production.

The hematopoietic stem or progenitor cells used to inoculate the cell culture may be derived from any source including bone marrow, both adult and fetal, cytokine or chemotherapy mobilized peripheral blood, fetal liver, bone marrow, umbilical cord blood, embryonic yolk sac, fetal liver, and spleen, both adult and fetal. A most advantageous biological source of such cells is umbilical cord blood. Bone marrow cells may be obtained from any known source, including but not limited to, ilium (e.g. from the hip bone via the iliac crest), sternum, tibiae, femora, spine, or other bone cavities.

For isolation of bone marrow from fetal bone or other bone source, an appropriate solution may be used to flush the bone, including but not limited to, salt solution, conveniently supplemented with fetal calf serum (FCS) or other naturally occurring factors, in conjunction with an acceptable buffer at low concentration, generally from about 5-25 mM. Convenient buffers include, but are not limited to, HEPES, phosphate buffers and lactate buffers. Otherwise, bone marrow may be aspirated from the bone in accordance with conventional techniques.

In those embodiments in which the HSCs are being expanded for autologous bone marrow transplantation, it is preferable that the initial inoculation population of HSCs is separated from any neoplastic cells prior to culture. Isolation of the phenotype (CD34⁺ Thy-1⁺ CD14⁻ CD15⁻) from multiple myeloma patients has been shown to reduce the tumor burden to less than 1 tumor cell per 10⁵ purified cells.

Selection of hematopoietic stem and progenitor cells can be performed by any number of methods, including cell sorters, magnetic separation using antibody-coated magnetic beads, packed columns; affinity chromatography; cytotoxic agents joined to a monoclonal antibody or used in conjunction with a monoclonal antibody, including but not limited to, complement and cytotoxins; and “panning” with antibody attached to a solid matrix, e.g., plate, or any other convenient technique.

The use of separation techniques include, but are not limited to, those based on differences in physical (density gradient centrifugation and counter-flow centrifugal elutriation), cell surface (lectin and antibody affinity), and vital staining properties (mitochondria-binding dye rhol23 and DNA-binding dye Hoechst 33342). Techniques providing accurate separation include but are not limited to, FACS, which can have varying degrees of sophistication, e.g., a plurality of color channels, low angle and obtuse light scattering detecting channels, impedance channels, and the like.

Such antibodies can be conjugated to identifiable agents including, but not limited to, enzymes, magnetic beads, colloidal magnetic beads, haptens, fluorochromes, metal compounds, radioactive compounds, drugs or haptens. Enzymes that can be conjugated to the antibodies include, but are not limited to, alkaline phosphatase, peroxidase, urease and beta-galactosidase. Fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies, see Haugland, Molecular Probes: Handbook of Fluorescent Probes and Research Chemicals (1992-1994). Metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxygenin, oxazalone, and nitrophenol. The radioactive compounds that can be conjugated or incorporated into the antibodies are known to the art, and include but are not limited to technetium 99 m (⁹⁹TC), ¹²⁵I and amino acids comprising any radionuclides, including, but not limited to, ¹⁴C, ³H and ³⁵S.

Other techniques for positive selection may be employed, which permit accurate separation, such as affinity columns, and the like. The method should permit the removal to a residual amount of less than about 20%, preferably less than about 5%, of the non-target cell populations.

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

It also is possible to enrich the inoculation population for CD34⁺ cells prior to culture, using for example, the method of Sutherland et al., 1992, Exp. Hematol. 20: 590 and that described in U.S. Pat. No. 4,714,680. Preferably, the cells are subject to negative selection to remove those cells that express lineage specific markers. Methods of negative selection are known in the art. As used herein, lineage-negative (LIN⁻) refers to cells lacking at least one marker associated with lineage committed cells, e.g., markers associated with T cells (such as CD2, 3, 4 and 8), B cells (such as CD10, 19 and 20), myeloid cells (such as CD14, 15, 16 and 33), natural killer (“NK”) cells (such as CD2, 16 and 56), RBC (such as glycophorin A), megakaryocytes (CD41), mast cells, eosinophils or basophils or other markers such as CD38, CD71, and HLA-DR. Preferably the lineage specific markers include, but are not limited to, at least one of CD2, CD14, CD15, CD16, CD19, CD20, CD33, CD38, HLA-DR and CD71. More preferably, LIN⁻ will include at least CD14 and CD15. Further purification can be achieved by positive selection for, e.g., c-kit⁺ or Thy-1⁺. Further enrichment can be obtained by use of the mitochondrial binding dye rhodamine 123 and selection for rhodamine⁺ cells, by methods known in the art. A highly enriched composition can be obtained by selective isolation of cells that are CD34⁺, preferably CD34⁺LIN⁻, and most preferably, CD34⁺ Thy-1⁺ LIN⁻. Populations highly enriched in stem cells and methods for obtaining them are well known to those of skill in the art, see e.g., methods described in PCT/US94/09760; PCT/US94/08574 and PCT/US94/10501.

Selection of progenitor cells or stem cells need not be achieved solely with a marker specific for the cells. By using a combination of negative selection and positive selection, enriched cell populations can be obtained.

Various techniques may be employed to separate cells by initially removing cells of dedicated lineage. Monoclonal antibodies are particularly useful for identifying markers associated with particular cell lineages and/or stages of differentiation. The antibodies may be attached to a solid support to allow for crude separation. The separation techniques employed should maximize the retention of viability of the fraction to be collected. Various techniques of different efficacy may be employed to obtain “relatively crude” separations. The particular technique employed will depend upon efficiency of separation, associated cytotoxicity, ease and speed of performance, and necessity for sophisticated equipment and/or technical skill. In a non-limiting example, using umbilical cord blood CD34⁺ cells are enriched (>95% purity) by Ficoll separation followed by isolation using CD34-specific monoclonal antibodies conjugated to immunomagnetic beads using conventional procedures.

The expansion methods of the invention generally requires inoculating a population of cells from a biological source such as umbilical cord blood into an expansion container and in a volume of a suitable medium such that the cell density is from at least about 5,000, preferably 7,000 to about 200,000 cells/mL of medium, and more preferably from about 10,000 to about 150,000 cells/mL of medium, and at an initial oxygen concentration of from about 2 to 20% and preferably less than 8%. In one embodiment, the initial oxygen concentration is in a range from about 4% to about 6%. In one aspect, the inoculating population of cells is derived from adult bone marrow and is from about 7,000 cells/mL to about 20,000 cells/mL and preferably about 20,000 cell/mL. In a separate aspect, the inoculation population of cells is derived from mobilized peripheral blood and is from about 20,000 cells/mL to about 50,000 cells/mL, preferably 50,000 cells/mL. In non-limiting example, 40,000 to 50,000 CD34⁺ cells/ml are used to start hematopoietic progenitor cell or hematopoietic stem cell culture from umbilical cord blood using a fully humidified incubator having 5% CO₂ and atmospheric O₂ (˜20%).

Any suitable expansion container, flask, or appropriate tube such as a 24 well plate, 12.5 cm² T flask or gas-permeable bag can be used in the method of this invention. Such culture-containers are commercially available from Falcon, Corning or Costor. As used herein, “expansion container” also is intended to include any chamber or container for expanding cells whether or not free standing or incorporated into an expansion apparatus such as the bioreactors described herein. In one embodiment, the expansion container is a reduced volume space of the chamber which is formed by a depressed surface and a plane in which a remaining cell support surface is orientated.

Various media can be used for the expansion of hematopoietic progenitor cells or stem cells prepared from a biological source such as umbilical cord blood. Illustrative media include Dulbecco's MEM, IMDM and RPMI-1640 that can be supplemented with a variety of different nutrients, growth factors, cytokines, etc. The media can be serum free or supplemented with suitable amounts of serum such as fetal calf serum or autologous serum. Preferably, if the expanded cells or cellular products are to be used in human therapy, the medium is serum-free or supplemented with autologous serum. One suitable medium is one containing Iscove's Modified Dulbecco's Medium (IMDM), effective amounts of at least one of a peptone, a protease inhibitor and a pituitary extract and effective amounts of at least one of human serum albumin or plasma protein fraction, heparin, a reducing agent, insulin, transferrin and ethanolamine. In a further embodiment, the suitable expansion medium contains at least IMDM and 1-15% fetal bovine serum. Other suitable media formulations are well known to those of skill in the art, see for example, U.S. Pat. No. 5,728,581. In particular embodiments the media is supplemented with at least one cytokine at a concentration from about 0.1 ng/mL to about 500 ng mL, more usually 10 ng/mL to 100 ng/mL. Suitable cytokines, include but are not limited to, c-kit ligand (KL) (also called steel factor (StI), mast cell growth factor (MGF), and stem cell factor (SCF)), IL-6, G-CSF, IL-3, GM-CSF, IL-1.alpha., IL-11 MIP-1alpha, LIF, c-mpl ligand/TPO, and flk2/flk3 ligand. (Nicola et al., 1979, Blood 54: 614-627; Golde et al., 1980, Proc. Natl. Acad. Sci. (USA) 77: 593-596; Lusis, 1981, Blood 57: 13-21; Abboud et al., 1981, Blood 58: 1148-1154; Okabe, 1982, J. Cell. Phys. 110: 43-49; Fauser et al., 1981, Stem Cells 1: 73-80). In advantageous embodiments the culture includes Flt-3 ligand, TPO, IL-3 and SCF. In one embodiment, the cytokines are contained in the media and replenished by media perfusion. Alternatively, when using a bioreactor system, the cytokines may be added separately, without media perfusion, as a concentrated solution through separate inlet ports. When cytokines are added without perfusion, they will typically be added as a 10-fold to 100-fold concentrated solution in an amount equal to one-tenth to 1/100 of the volume in the bioreactors with fresh cytokines being added approximately every 2 to 4 days. Further, fresh concentrated cytokines also can be added separately in addition, to cytokines in the perfused media.

The population is then cultured under suitable conditions such that the cells condition the medium. Improved expansion of purified stem cells may be achieved when the culture medium is not changed, e.g., perfusion does not start until after the first several days of culture.

In most aspects, suitable conditions comprise culturing at 33° C. to 39° C., and preferably around 37° C., oxygen concentration that is preferably 20% and carbon dioxide concentration that is 5%, for at least 6 days and preferably from about 7 to about 10 days and most particularly 9 days, to allow release of autocrine factors from the cells without release of sufficient waste products to substantially inhibit stem cell expansion. In particular embodiments, hematopoietic progenitor cells from umbilical cord blood are grown for around 9 days and hematopoietic stem cells from umbilical cord blood cultured for around 9 days.

Use of Expanded Cultures of Hematopoietic Progenitor or Stem Cells

Ex vivo expanded hematopoietic progenitor cells or stem cells can be used in various clinical applications for preventing, ameliorating numerous diseases. Most particularly, provided herein are methods for preventing or treating leukocytopenia or neutropenia in cancer chemotherapy patient, or radiation or chemotherapy recipients with leukemia or lymphoma. As set forth herein, the invention provides compositions and methods for treating these aspects of clinical presentation of disease.

A particular use for the genes of the present invention is autologous bone marrow transplants for individuals suffering from bone marrow aplasia or myelosuppresion such as that seen in response to radiation therapy or chemotherapy. High dose or lethal conditioning regimens using chemotherapy and/or radiation therapy followed by rescue with allogeneic stem cell transplantation (allo-SCT) or autologous stem cell transplantation (ASCT) have been the treatments of choice for patients with a variety of hematologic malignancies and chemosensitive solid tumors resistant to conventional doses of chemotherapy. A common source of stem cells for such procedures has been the bone marrow and recently, peripheral blood stem cells (PBSC) have also been used. Alternative sources of progenitor or stem cells or both such as umbilical cord blood, which has advantages in terms of its availability and capacity to provide hematopoietic stem cells non-invasively, have not been exploited due to the lack of sufficient hematopoietic progenitor cells or stem cells from this source. The methods of the present invention can be used to expand progenitor or stem cell populations or both from sources such as umbilical cord blood, to enhance the number of hematopoietic progenitor or stem cells thereby improving clinical outcomes for such patients.

Expanding umbilical cord blood stem cell populations is an example of ex vivo therapy which employs HSC populations that can be introduced into a patient who has been treated with a therapeutic modality that suppresses or ablates endogenous stem cell function. Cells from a source such as umbilical cord blood are expanded according to the methods of this invention and introduced into the patient. In ex vivo therapy, cells from the patient are removed and maintained outside the body for a period of time. During this period, the cells are expanded according to the methods of the present invention and then reintroduced into the patient. In certain embodiments, the patient will serve as his/her own bone marrow donor. The methods of this invention can be used in conjunction with cancer therapy in which a normally lethal dose of irradiation or chemotherapy may be delivered to the patient to kill tumor cells, and the bone marrow repopulated with the patients own cells that have been maintained and expanded ex vivo. Reintroducing into the patient a HSC population that has been grown according to the methods of the present invention will be particularly useful in patients that are undergoing chemotherapy or radiation that destroys the bone marrow of the patient.

Thus, the present invention contemplates a method of treating a human patient having a pathogenic cell disease which requires administration of hematopoietic progenitor cells or stem cells or both expanded according to the methods described herein. Preferably, the stem cells are peripheral blood stem cells, umbilical cord blood stem cells or bone marrow stem cells. The HPCs or HSCs expanded according to the methods provided herein may be used in the treatment of any clinical diseases involving hematopoietic dysfunction or failure, either alone or in combination with other lymphokines or chemotherapy. Such disorders include leukemia and white cell disorders in general. The HPCs or HSCs can be used in induced forms of bone marrow aplasia or myelosuppression, in radiation therapy, accidental exposure to radiation, or chemotherapy-induced bone marrow depletion, wound healing, burn patients, and in bacterial inflammation, among other indications known in the art.

Pathogenic cell diseases treatable with the methods include malignant diseases such as chronic myelogenous leukemia, acute myelogenous leukemia, acute lymphoblastic leukemia, non-Hodgkin's lymphoma, myelodysplastic syndrome or multiple myeloma. The malignant disease may also be a solid tumor as in testicular cancer or various types of brain tumor. The disease being treated alternatively may be a non-malignant diseases such as sickle cell anemia, beta-thalassemia major, Blackfan Diamond Anemia, Gaucher's anemia, Fanconi's anemia or AIDS. The non-malignant disease may also be an autoimmune disease.

Additionally, the invention provides the results of global microarray data that revealed a set of differentially expressed genes linked with HSC expansion rather than HSC maintenance or loss, permitting a cell population to be assayed to identify and quantify hematopoietic stem cells in an ex vivo expanded cell culture. Epigenetic changes likely play a key role in governing inflammatory signals specifically involved in HSC expansion and maintenance. Epigenetic changes such as DNA methylation and histone acetylation are important for modifying gene expression and ultimately the function of HSC (Araki et al., 2006, Exp Hematol. 34: 140-149; Jones &Takai, 2001, Science 293: 1068-1070; Marks et al., 2000, J. Nat. Cancer Inst. 92: 1210-1216; Reik et al., 2001, Science 293: 1089-1093). Histone acetylation has also been suggested to have a profound effect on the normal transition from a fetal to an adult hematopoietic cellular differentiation program during ontogeny (Agata et al., 2001, J Exp Med. 193:873-880). As set forth herein, epigenetics play a role in silencing genes likely involved in regulation of HSC maintenance and expansion in culture. This epigenetic process can be circumvented by use of CMA in culture which can activate genes by direct or indirect mechanisms resulting in distinct HSC fate choices: expansion or maintenance. The gene expression pattern in expanded CD34⁺ cells at least in part results from epigenetic modifications which include changes in histone acetylation and promoter CpG sites methylation. The use of additional CMAs in culture expands transplantable HSC and correlates with CMA-induced changes in gene expression and HSC functions as defined by in vivo hematopoietic reconstitution. Differential gene expression from global microarray studies using CMA-expanded CD34⁺ cells in conjunction with their in vivo HSC functions revealed distinct gene expression patterns associated with functional HSC expansion or maintenance. However, 5azaD/TSA is more efficacious than VPA in expanding in vivo repopulating HSC. VPA-expanded CB cells are capable of reconstituting both myeloid and lymphoid blood lineages in primary hosts but fail to engraft secondary hosts. These results demonstrated that while sequential ex vivo treatment of CD34⁺ cells with 5azaD/TSA expanded transplantable HSC, VPA treatment only permitted HSC maintenance as shown by in vivo transplant studies. Addition of CMAs to the culture is associated with increased transcript levels of polycomb group genes including Ezh2 and Bmi1, which are known to regulate HSC self-renewal (Araki et al., 2007, Blood 109:3570-3578; Iwama et al., 2004, Immunity 21:843-851; Kamming a et al., 2006, Blood 107: 2170-2179; Rizo et al., 2009, Blood 114: 1498-1505). Similarly expression of genes known to be responsible for HSC maintenance, including GATA2, and CDK inhibitor P21, are increased in CMA-expanded CD34⁺ cells. Although expression of some differentiation associated transcription factors were detected in 5azaD/TSA-expanded CD34⁺ cells it was not statistically significant. Expression of differentiation associated genes may be due to the presence of a small subpopulation of relatively mature CD34⁺ cells or aberrant gene expression in relatively primitive HSC as described previously (Akashi et al., 2003, Blood 101: 383-389).

Distinct sets of genes associated with HSC expansion or maintenance were identified from differential gene expression profiles based on the type of CMAs used in culture and the in vivo function of the expanded HSC. It is important to emphasize that although CD34⁺ cells in CB represents less than 1% of total nucleated cells (TNC) and are enriched for HSCs, they remain a relatively heterogeneous population. Purified, expanded CD34⁺ cell populations were used for analysis of differential gene expression from global microarray analysis following culture with different CMA treatment histories in comparison to primary uncultured CD34⁺ cells with each having distinct in vivo functional outcomes namely expansion, maintenance or loss of transplantable HSC. This approach can potentially compensate for the limitations of heterogeneity of the CD34⁺ cell population and may also facilitate identification of a gene signature with functional relevance. No difference in the transcript levels of candidate genes implicated in HSC expansion including HoxB4, Ezh2 and Bmi1 were observed regardless of whether CD34⁺ cells were expanded in 5azaD/TSA or VPA. However, xenotransplant studies indicate that, unlike 5azaD/TSA, VPA treatment of CD34⁺ cells allows for maintenance rather than expansion of transplantable HSC. These observations raise questions whether the roles of these genes are confined exclusively to HSC expansion, or if expansion and maintenance are distinct gene functions for biologically separable processes. It may be that CMAs result in both direct and indirect effects early in the culture which gives rise to a transcriptome state which promotes HSC expansion. For instance, as a direct result of CMA treatment significant reduction in methylation levels were detected in S100A8, Cyp11A1 and GATA1 gene promoter sites corresponding with their increased transcript levels in CD34⁺ cells expanded in 5azaD/TSA. Furthermore, heat map analysis highlights a distinct differential gene expression pattern between CD34⁺ cells possessing or lacking in vivo marrow repopulation potential. The data also shows that CpG islands near the promoter areas of genes including HoxB4 and GATA2 do not have significant changes in methylation suggesting that their increased transcript levels in CMA-expanded CD34⁺ cells are likely due to indirect effects and not directly related to epigenetic changes. Interestingly the level of HoxB4 was significantly higher than control in CD34⁺ cells expanded with either 5azaD/TSA or VPA. Furthermore, differential gene expression data indicate that GATA1 and GATA2 are exclusively associated with HSC maintenance function, but the promoter of GATA1 and not GATA2 was hypomethylated in 5azaD/TSA expanded CD34⁺ cells. However, GATA1 expression was higher in both VPA and 5azaD/TSA expanded CD34⁺ cells despite a lack of changes in methylation in VPA-expanded CD34⁺ cells, which is consistent with indirect effects of VPA treatment. It is likely that exposure of CD34⁺ cells to CMA, in particular 5azaD/TSA, results in activation of genes which work cooperatively and likely promote symmetric self-renewing HSC divisions during ex vivo culture as evident by the net expansion of SRC shown by xeno-transplantation studies. Differential gene expression from global microarray studies as shown here may help in identifying such gene networks. Temporal effects, including early epigenetic modifications, may lead to changes in transcription factor expression, which directly or indirectly promote symmetric or asymmetric HSC divisions, ultimately resulting in HSC expansion or maintenance.

Identifying a gene expression signature linked with in vivo blood regeneration capacity has significant clinical applications. In order to expand HSC, both expansion and maintenance genes are likely necessary. It is interesting to note that the transcript levels of genes generally implicated in HSC self renewal and differentiation (e.g. HoxB4, GATA1) did not allow for discrimination between CD34⁺ cells expanded in 5azaD/TSA or VPA. In contrast, expression profiling data indicate differential expression of gene transcripts such as Cyp11A1, Alox5, and F2RL2 was observed between 5azaD/TSA and VPA expanded CD34⁺ cells and may serve as biomarkers indicating successful expansion or maintenance of transplantable HSC. Interestingly S100A8, whose expression was higher in CMA-expanded CD34⁺ cells, has been shown to be a toll like receptor 4 (TLR4) agonist (Ehrchen et al., 2009, J. Leukoc. Biol. 86:557-566). Since the lipopolysaccharide receptor TLR4 has been detected in human CD34⁺ cells, S100A8 can play a more direct role in HSC expansion potentially mediated by signals involving inflammatory responses. TLR4 has been shown to play a role in maintenance and proliferation of endothelial progenitor cells (He et al., 2010, J. Cell Biochem. 111: 179-186). This is consistent with systemic infection influencing HSC cycling and the role of interferon as a positive regulator of HSC proliferation (Baldridge et al., 2010, Nature 465: 793-797). The demethylation of CpG sites corresponding with higher transcript levels of inflammation/stress associated genes including S100A8 and Cyp11A1 as well as the presence of an increase in inflammatory mediators such as, LTB4 in CMA-expanded cultures is consistent with at least a partial role for these molecules in HSC expansion and maintenance. LTB4 is synthesized from arachidonic acid by dual action of Alox5 gene product, 5-lipooxygenase and LTA4 hydrolase, further supporting possible involvement of signals involving inflammation/lipid metabolism potentially governing HSC fate choices in culture. In summary, expansion of HSC is associated with transient DNA hypomethylation and histone hyperacetylation. Epigenetic changes following ex vivo treatment of CD34⁺ cells using CMAs can trigger a transient inflammatory response activating genes contributing to HSC expansion or maintenance.

Each reference described and/or cited herein is incorporated by reference in its entirety.

The following examples are provided for the purpose of illustration and are not intended to limit the scope of the present invention.

### EXAMPLES

### Methods

### Isolation and Culture of CD34+ Cells

Freshly collected human CB was obtained from the New York Blood Center (New York, N.Y.) according to Institutional Review Board guidelines. Low density CB cells (<1.077 g/ml) were obtained by density centrifugation on Ficoll-Paque PLUS (GE Healthcare Bio-Sciences AB, Uppsala, Sweden), from which CD34⁺ cells were immunomagnetically isolated by the MACS CD34 progenitor isolation kit (Miltenyi Biotech, Inc., Auburn, Calif.) as described in Araki et al., 2009, Exp Hematol. 37: 1084-1095). The purity of isolated CD34⁺ cells routinely ranged from 90-99%.

CD34⁺ cell expansion culture with or without CMA was carried out as described (Araki et al., 2009, Id.; Araki et al. 2006, Exp Hematol. 34: 140-149; Araki et al, 2007, Blood 109: 3570-3578.). All other HDAC inhibitors including 1 mM VPA (Sigma), TSA (5 ng/mL), NA (5 mM) or 5 μM of Suberoylanilide hydroxamic acid (SAHA; BioVision, Inc. Mountain View, Calif.) was added twice at 0 hr and after changing media at 48 hrs of culture. HDAC inhibitors were added only once (48 hr) when used in combination with a DNMT inhibitor as described (Araki et al., 2009, Id.; Araki et al., 2006, Id.; Himburg et al., 2010, Nat. Med. 16: 475-482).

### Flow Cytometric Analysis

Flow cytometric analysis was carried out as described (Araki et al., 2009, Id.; Araki et al. 2006, Id.; Araki et al, 2007, Id.). All antibodies were purchased from BD Bioscience (San Jose, Calif.). All analyses were paired with the corresponding matched isotype control and at least 10,000 live cells were acquired for each analysis (CellQuest software, Becton Dickinson).

### CFC and CAFC Assays

Colony-forming cell (CFC) were assayed by plating 5×10² cells per dish in semisolid media and were counted after 14 days as described (Araki et al., 2009, Id.). The number of cobble stone area-forming cell (CAFC) was quantitated in primary and expanded CD34+ cells by plating in limiting dilution onto irradiated M2-10B4 stromal cells as described (Araki et al., 2009, Id.; Conneally et al., 1997, Proc. Natl. Acad. Sci. USA. 94: 9836-9841; Taswell, 1981, J. Immunol. 126: 1614-1619).

### NOD/SCID Assays

Immunodeficient nonobese diabetic/ltsz-scid/scid (NOD/SCID) mice were purchased from Jackson Laboratories (Bar Harbor, Me.) and transplantation assays were performed as described (Araki et al., 2009, Id.; Araki et al., 2007, Id.; Taswell, 1981, Id.). Equal number of primary CD34⁺ cells or the progeny of an equal initial number of CD34⁺ cells following culture were injected per mouse intravenously. Mice were sacrificed 8 weeks after transplantation, and bone marrow (BM) cells from each mouse were analyzed by flow cytometry to detect multilineage human cell engraftment.

For secondary transplantation two-thirds of unfractionated bone marrow (BM) of each primary recipient mouse which showed multilineage human engraftment after transplantation was injected into secondary recipients without further re-isolation of human cells.

### RNA Preparation and Real-Time RT-PCR

RNA preparation and real time quantative PCR assays were performed using SYBR green dye (Life Technologies; Carlsbad, Calif.) and an ABI 7500 Fast Real-Time PCR system (Life Technologies) were used to quantitate gene expression in reverse-transcribed mRNA as described (Araki et al., 2007, Id.). Gene expression was calculated by relative quantitation, and all results were normalized to expression of GAPDH. The primer sequences used in real time RT-PCR assays are shown in Table 1.

TABLE 1
Sequence of primers used for real time quantitative PCR analyses.

| Gene | | |
|---|---|---|
| Name | Forward Primer (SEQ ID No.) | Reverse Primer (SEQ ID No.) |
| GAPDH | TGCACCACCAACTGCTTAGC (1) | TCTTCTGGGTGGCAGTGATG (2) |
| c-Myc | CAGCAGCGACTCTGAGGAGGAACA (3) | GCCTCCAGCAGAAGGTGATCCAGA (4) |
| HoxB4 | ACCTCGACACCCGCTAACAAATGA (5) | AATGGGCACGAAAGATGAGGGAGA ( 6) |
| Bmi1 | TGTGTGTGCTTTGTGGAGGGTACT (7) | TGCTGGTCTCCAGGTAACGAACAA ( 8) |
| P21 | GGTCTGACCCCAAACACCTTC ( 9) | AACGGGAACCAGGACACATG (10) |
| Ezh2 | CAGTTTGTTGGCGGAAGCGTGTAA (11) | AGGATGTGCACAGGCTGTATCCTT (12) |
| GATA1 | ACACCAGGTGAACCGGCCACT (13) | CCTTCGGCTCCTCCTGTGCC (14) |
| GATA2 | ATTGTCAGACGACAACCACCACCT(15) | TTCCTTCTTCATGGTCAGTGGCCT (16) |
| cEBPa | CCTTGTGCAATGTGAATGTGC (17) | CGGAGAGTCTCATTTTGGCAA (18) |
| Alox5 | TGTGGGAAGCCATCAGGACGTTCA (19) | CCGCATGCCGTACACGTAGACATC (20) |
| S100A8 | GGGATGACCTGAAGAAATTGCTA (21) | TGTTGATATCCAACTCTTTGAACCA (22) |
| Pu.1 | AACGCCAAACGCACGAGTATTACC (23) | TGAAGTTGTTCTCGGCGAAGCTCT (24) |
| Cyp11A1 | TGGCATCTCCACCCGCAGTC (25) | GAGCTTCTCCCTGTAAATCGGGCC (26) |
| Col14A1 | GTGTGGCCGATGCAGATTACTCGG (27) | CCACTGGACAGGTTGCTGATGCTG (28) |
| F2RL2 | AGGCTTCCATTTGCTGCTGACACA (29) | TCCATGCCACTCTGACAAAAAGTGGG (30) |
| MPEG1 | CAACCAGACGAGGATGGCCACCTA (31) | GACTGCTCTGGCTGTCTTGGAGGA (32) |
| ALDH1A1 | GGCCGCAAGACAGGCTTTTCAGAT (33) | ATTGACTCCATTGTCGCCAGCAGC (34) |
| BMPR1A | ACTGCCCCCTGTTGTCATAGGTCC (35) | ACGTCTGCTTGAGATGCTCTTGCA (36) |

Primers were designed using the PrimerQuest ^{SM} software (Integrated DNA Technologies, Coralville, IA). All primers are in 5′ to 3′ direction.

### Microarray Studies

Global gene expression microarray studies utilizing Affymetrix U133 Plus 2.0 array (Affymetrix, Santa Clara, Calif.) were performed in collaboration with the UCLA Clinical Microarray Core as described with minor modifications (Reeves et al., 2010, BMC Cancer 10: 562.). Briefly, total RNA was extracted from either unmanipulated primary CD34⁺ cells or enriched CD34⁺ cells (>90% purity) after expansion culture with or without CMA using TRIzol (Life Technologies, NY, USA) followed by Qiagen column purification (Qiagen, Valencia, Calif., USA). The samples for microarray studies included control, 5azaD/TSA, VPA, and TSA/5azaD expanded as well as primary unmanipulated CD34⁺ cells. All microarray studies were repeated in triplicate except for TSA/5azaD expanded CD34⁺ cells, which was carried out in duplicate. Each of the replicate samples represented a pool of 4 to 8 independent CB units. RNA integrity was evaluated by an Agilent 2100 Bioanalyzer (Agilent Technologies; Palo Alto, Calif.) and purity and concentration was determined by using NanoDrop 8000 (NanoDrop, Wilmington, Del., USA).

Subsequent data analyses were performed using Partek Genomics Suite with the CEL files obtained from GCOS. The data were normalized using RMA algorithm. The significant genes were selected at >2-fold, and p<0.05. Global functional analyses, network analyses and canonical pathway analyses were performed using Ingenuity® Pathway Analysis 8.6 (Ingenuity Systems, Redwood City).

### Chromatin Immunoprecipitation (ChIP) Assays

The ChIP assay was carried out using a commercial Assay kit using the manufacturer's protocol (Millipore; Billerica, Mass.) as described (Sankar et al., 2008, Oncogene 27: 5717-5728). Briefly, after 72 hours of culture CD34⁺ cells were crosslinked with formaldehyde and the cell pellet was lysed and sonicated to produce genomic fragments and immunoprecipitated using anti-acetyl-Histone H4 antibody (Millipore). The precipitated chromatin bound DNA was PCR amplified using the primers for specific genes as shown in Table 2. Amplified PCR fragments were then analyzed on a 1.2% agarose gel.

TABLE 2
Immunoprecipitated DNA was PCR amplified using the following
primers specific for the genes and regions indicated

| Gene | Region | Forward Primer | Reverse Primer |
|---|---|---|---|
| Name | Amplified | (SEQ ID No.) | (SEQ ID No.) |
| HoxB4 | −537 to | GCGAAGTCTCCCCGAATTAGTG | GTCTCTATGGGGAGTTAGGTTACT |
| | −727 | (37) | (38) |
| BMI-1 | −240 to | CAGCAACTATGAAATAATCGTAG | TCCGCCTCCGCCTCGACCTCCAAC |
| | −506 | (39) | (40) |
| Pu.1 | −4 to | GACTATCTCCCAGCGGCAGGCC | CCGGGCTCCGAGTCGGTCAGATC |
| | −224 | (41) | (42) |
| GATA2 | −55 to | TCGGACTGACCACGTTCAGCGGTGAAGG | AAGCCAGCCAATCAACGCCGCG |
| | −435 | (43) | (44) |
| GAPDH | +528 to | ACAGTCCATGCCATCACTGCC | GCCTGCTTCACCACCTTCTTG |
| | +793 | (45) | (46) |

All primers are in 5′ to 3′ direction.

### DNA Methylation Analysis

DNA methylation analysis was performed by EpigenDx (Worcester, Mass.) using quantitative pyrosequencing and the PSQ-HS96 system according to standard procedures (Warren et al., 2010, Cell Stem Cell. 7: 618-630). Primers were developed for the CpG sites near the promoter area as detailed in Table 3. Briefly, genomic DNA was isolated from primary or CMA expanded enriched CD34⁺ cells using the Blood and Cell Culture DNA kit (Qiagen, Valencia Calif.). Genomic DNA was bisulfite treated by an EZ Methylation Kit (Zymo Research, Irvine, Calif.), and biotinylated gene specific primers or long interspersed nucleotide element 1 (LINE-1) primers were used to amplify regions of interest for analysis as described (Bhatia et al., 1997, J Exp Med. 186: 619-624).

TABLE 3
Pyrosequencing of CpG sites from gene
promoter regions as indicated below

| Gene | Region Analyzed |
|---|---|
| Ezh2 | −253 to −57 |
| GATA1 | −203 to −186 |
| GATA2 | −366 to −216 |
| STAT3 | −168 to +98 |
| HoxB4 | −178 to −44 |
| p21 | −117 to +61 |
| pu.1 | −296 to −198 |
| Alox5 | +44 to +98 |
| S100A8 | −1010 to −851 |
| CYP11A1 | −62 to +113 |

Genomic DNA was isolated from primary, 5azaD/TSA or VPA expanded CD34⁺ cells following culture using the Blood and Cell Culture DNA kit (Qiagen, Valencia Calif.). Genomic DNA was bisulfite treated by an EZ Methylation Kit (Zymo Research, Irvine, Calif.).

### Measurement of LTB4 by ELISA

The concentration of LTB4 in conditioned media was measured by an acetylcholine esterase competitive enzyme immunoassay following the manufacturer's instructions (Cayman Chemical Co. Ann Arbor, Mich.). Absorbance at 405 nm was read using ELx 800, instrument (Bio Tek Instruments Inc.). Results were calculated using mean values of triplicate wells with standard errors (SE).

### Statistical Analysis

The statistical significance (P<0.05) between the groups were determined using the Student t test.

### Example 1

### Valproic Acid Results in Expansion of Primitive CD34⁺CD90⁺ Cells

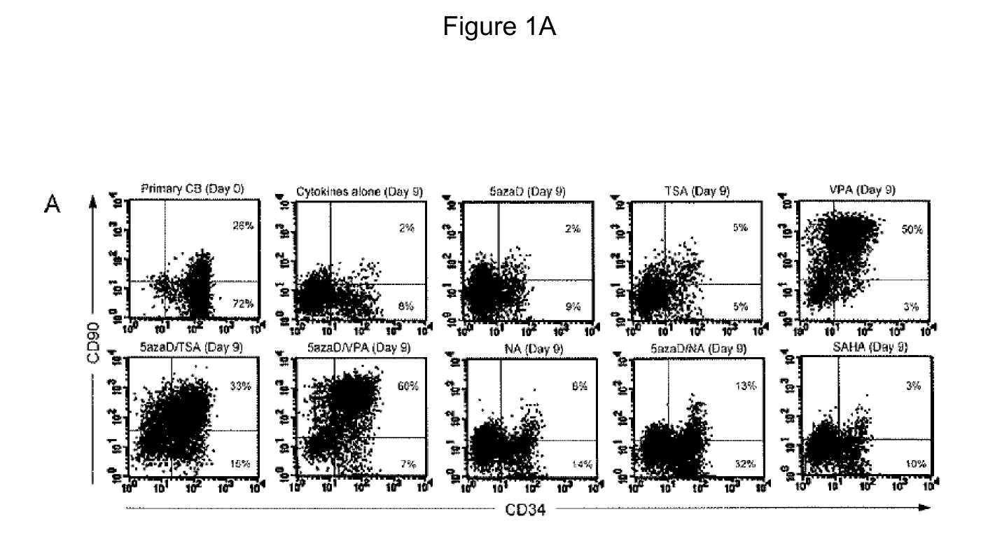

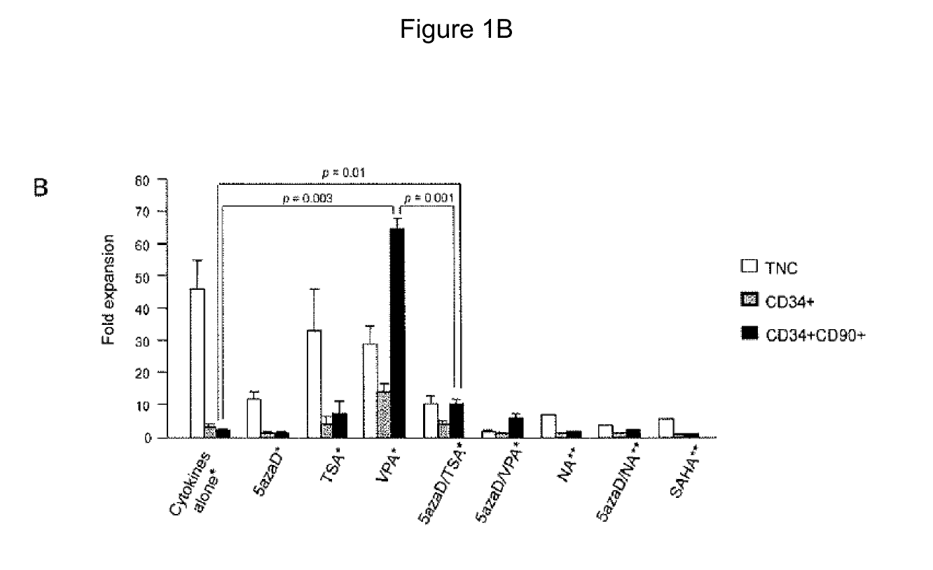

In order to compare the effects of various chromatin modifying agents (CMAs) on the degree of expansion of cord blood-derived, primitive subpopulation of CD34⁺ cells, valproic acid (VPA), trichostatin A (TSA), nicotinic acid (NA), suberoylanilide hydroxamic acid (SAHA) or 5-aza-2′-deoxycytidine (5azaD) as single agents or in combination was tested in vitro. The results of fluorescence-activated cell sorting experiments performed on cord blood-derived CD34⁺/CD90⁺ cells treated with CMA agents is shown in FIG. 1A. Briefly, the cells were cultured for 9 days and 2% of cord blood cells that were exposed to cytokines (SCF, Flt3-ligand, TPO and IL-3) alone were found to co-express CD34⁺ and CD90⁺, while 2% (5azaD), 5% (TSA), 6% (NA), 13% (the combination of 5azaD and NA) and 3% (SAHA) of the cells in the cultures receiving a combination of cytokines and chromatin modifying agents co-expressed CD34⁺ and CD90⁺.

Specific combinations and sequences of administration of CMAs displayed more dramatic expansion of primitive CD34⁺ CD90⁺ cells. For instance, cultures containing VPA (42.2%±13.5%), 5azaD/TSA (28.2%±3.6%), or 5azaD/VPA (52.4%±9.5%) contained a relatively higher percentage of primitive CD34⁺CD90⁺ cells (FIG. 1A). The combination of VPA and cytokines led to a 64.6±3.7-fold expansion of primitive CD34⁺CD90⁺ cells numbers as compared to a 1.2±0.5-fold (5azaD), 7.8±3.5-fold (TSA), 10.7±1.1-fold (5azaD/TSA), 6.4±1.3-fold (5azaD/VPA), 2.1-fold (NA), 2.2-fold (5azaD/NA) or 1.2-fold (SAHA) expansion in cultures receiving cytokines with various CMAs (as shown in FIG. 1B). These results indicate that while the combination of 5azaD and TSA in the culture results in 10.7-fold expansion of CD34⁺CD90⁺ cells, addition of VPA resulted in a much higher (65-fold) expansion of CD34⁺CD90⁺ cells (P=0.001, FIG. 1B). However, when VPA was added following 5azaD administration, the fold expansion of CD34⁺CD90⁺ cells (FIG. 1B) was much lower among the various CMAs tested despite the highest percentage (52.4%±9.5%) of CD34⁺CD90⁺ cells (FIG. 1A) occurring in cultures treated with the combination of 5azaD and VPA. VPA treatment alone provided the maximal expansion of CD34⁺ and CD34⁺CD90⁺ cells using a single CMA (42.2%±13.5%). NA and SAHA as single agents or in combination with 5azaD did not promote the expansion of CD34⁺CD90⁺ cells (FIG. 1B). These data indicated that among the CMAs tested, the most primitive CD34⁺CD90⁺ cells are expanded in the presence of 5azaD/TSA and VPA. The highest expansion of total nucleated cells (TNC) was observed in culture lacking CMA while addition of CMA resulted in relatively higher number of CD34⁺ cells and more primitive CD34⁺CD90⁺ cells but lower TNC.

### Example 2

### Functional Potency of CMA Expanded Grafts

### VPA Results in Maintenance While 5azaD/TSA Expands Transplantable HSC

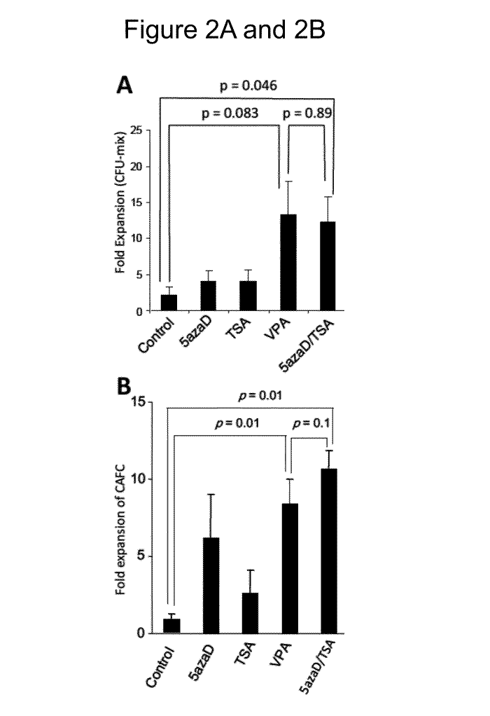

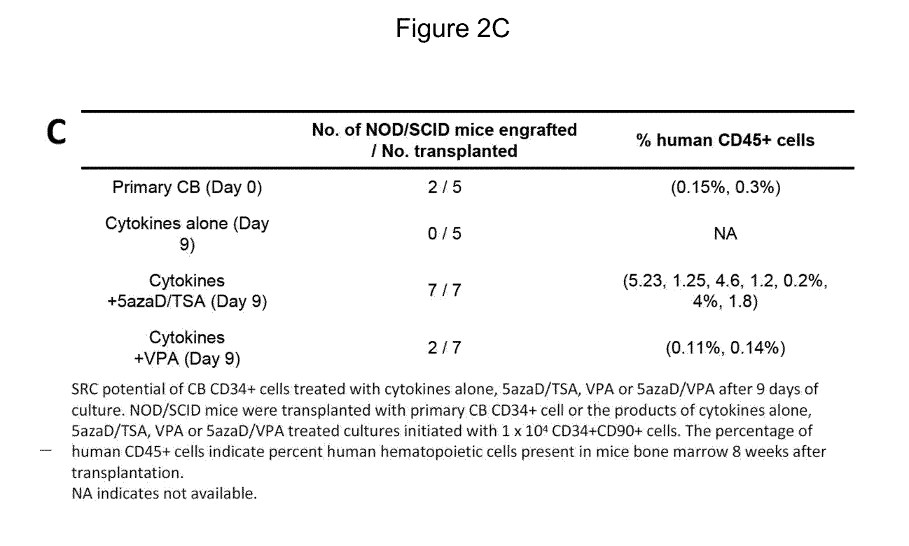

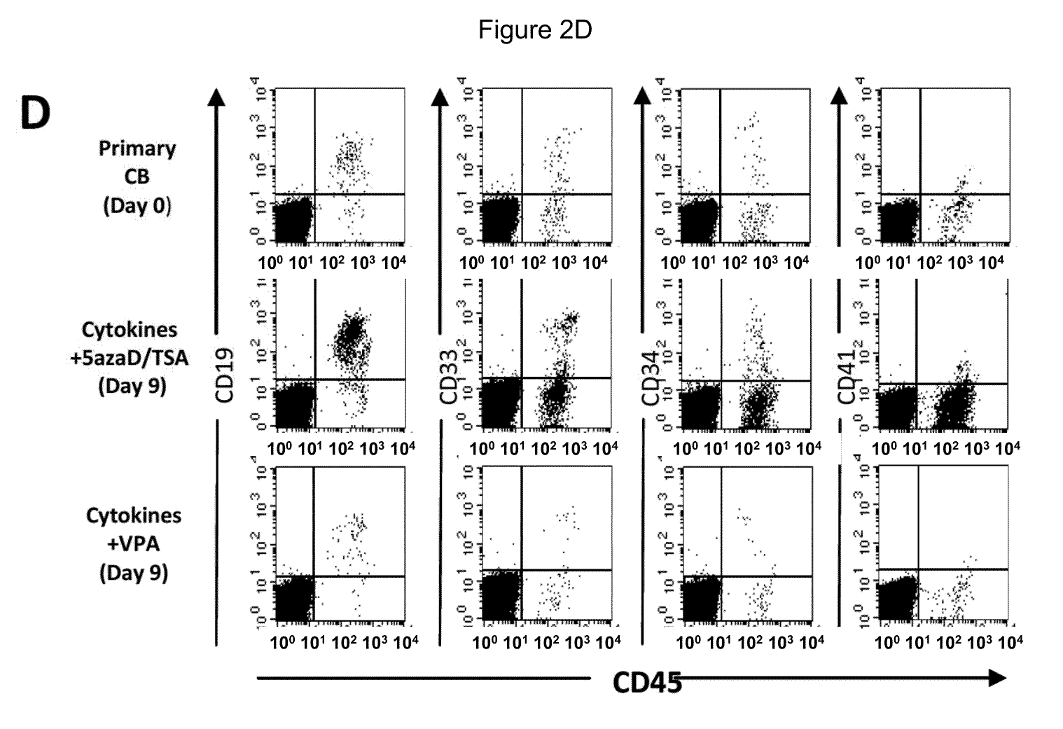

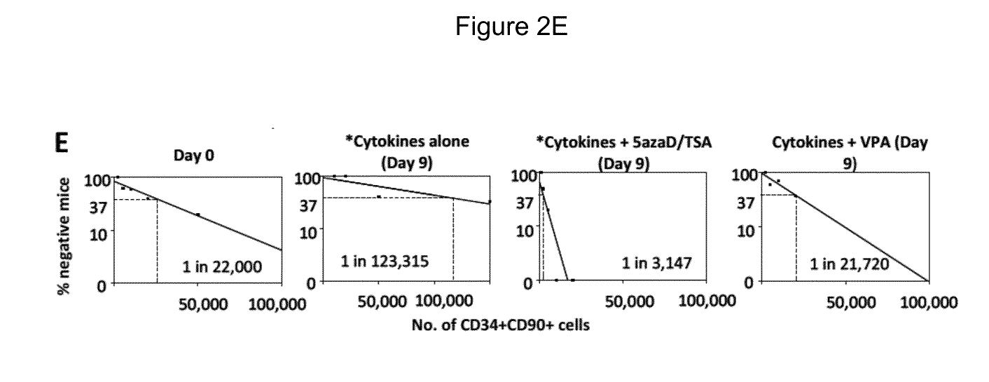

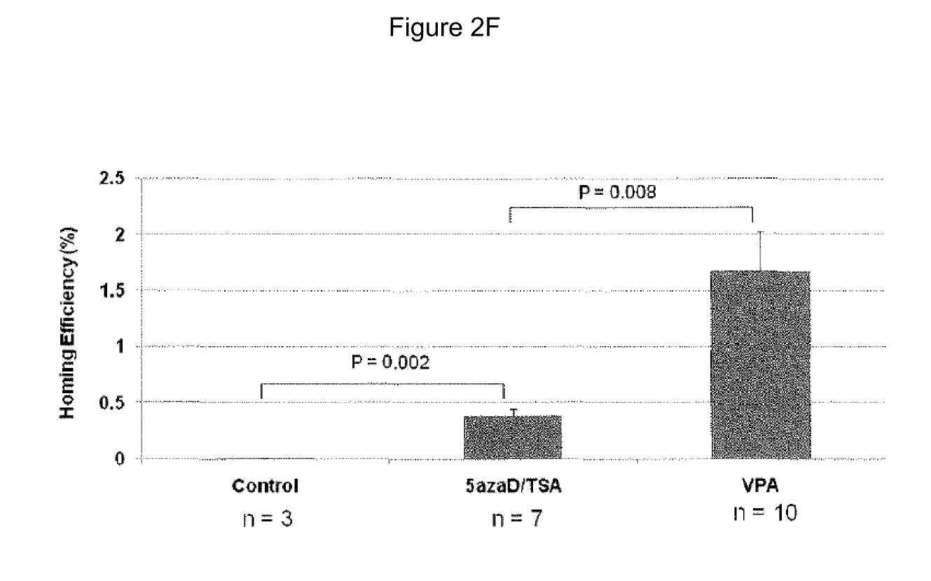

Because 5azaD/TSA used in combination and VPA alone displayed the highest expansion of the absolute number of primitive CD34⁺CD90⁺ cells following culture, subsequent experiments used VPA or 5azaD/TSA in the culture media instead of cytokines alone. In order to determine the a correlation between expansion of CD34⁺CD90⁺ cells and their functional potential as pluripotent hematopoietic stem cells, in vitro functional assays were performed, including assessment of colony-forming cells (CFC), a short-term assay, and cobblestone-area forming cells (CAFC), a long-term assay. It had been previously shown that an increase in CD34⁺CD90⁺ cells following 5azaD/TSA treatment was accompanied by retention of the ability of these cells to produce CFC and CAFC (Araki et al., 2006, Exp Hematol. 34: 140-49; Araki et al., 2007, Blood 109: 3570-78; Araki et al., 2009, Exp Hematol. 37: 1084-95). As can be seen in FIG. 2A, cultures receiving cytokines with VPA or cytokines with 5azaD/TSA had the highest degree of expansion of primitive CFU-mix colonies (13.3±4.7 and 12.4±3.4-folds respectively). The plating efficiency (PE) of CFC for VPA and 5azaD/TSA expanded cells was 15.27%±3.66% and 16.7%±2.5%-respectively (Table 4). In contrast the plating efficiency of CFC for CB cells expanded in the absence of CMA (control) was only 4.67%±0.55%. VPA and 5azaD/TSA-expanded cultures had the highest degree of expansion of long term (5 weeks) CAFC (8.4±1.6-fold and 10.5±1.5-fold respectively; FIG. 2B). However the CAFC frequency of 5azaD/TSA expanded cells was almost 4 times higher than VPA expanded cells as determined by limiting dilution analyses (52.7±10.37 vs. 14.67±0.74, Table 4). Expansion of CD34⁺CD90⁺ cells following 5azaD/TSA, but not VPA, treatment, correlated with their functional potential, as demonstrated by both expansion of short-term colony-forming cells (CFC) and long-term cobblestone area-forming cells (CAFC), respectively. However, the 65-fold expansion of CD34⁺CD90⁺ cells achieved with VPA treatment yielded relatively lower expansion of CFCs and CAFCs in contrast to CD34⁺ cells expanded using 5azaD/TSA. Although the difference in CD34⁺CD90⁺ cell expansion between TSA alone and 5azaD/TSA, or TSA and 5azaD/VPA is not statistically significant, the functional capacity of 5azaD/TSA-expanded cells is much superior to that of TSA-expanded cells.

TABLE 4
Effects of CMA on HSC Phenotype and Function

| | CD34+CD90+ | CFC PE | CAFC |
|---|---|---|---|
| | (%) | (%) | Frequency/10⁴ cells |
| Cytokines | 1.17 ± 0.32 | 4.67 ± 0.55 | 1.10 ± 0.64 |
| Alone | | | |
| 5azaD Alone | 2.33 ± 0.60 | 8.70 ± 1.37 | 21.73 ± 14.57 |
| TSA Alone | 5.60 ± 0.29^{¤} | 4.97 ± 0.39 | 6.39 ± 4.81 |
| VPA Alone | 42.17 ± 13.45* | 15.27 ± 3.66^{‡‡} | 14.67 ± 0.74^{§} |
| 5azaD/TSA | 28.17 ± 3.61^{†} | 16.70 ± 2.50^{•} | 52.70 ± 10.37 |
| 5azaD/VPA | 64.7 ± 2.2 | N/A | N/A |

The effects of various CMA treatments on the co-expression of CD34 and CD90 surface antigens, Colony Forming Cell Plating Efficiency (CFC PE), and Cobblestone Area Forming Cell Frequency (CAFC Frequency). Results are expressed as mean ± SEM of three independent experiments; p was calculated by Student's T Test and is relative to Cytokines Alone.
^{§}p = 0.0001;
^{¤}p = 0.0005,
^{‡}p = 0.001,
^{†}p = 0.002,
**p = 0.006, p = 0.008,
^{•}p = 0.009,
*p ⁼ 0.04;
^{‡‡}p = 0.05,
N/A: not applicable.

Furthermore, hematopoietic stem cell potency of CMA-treated cord blood cells was evaluated in vivo by assaying hematopoietic repopulation potential of CD34⁺ cells expanded with various CMAs. In these experiments, non-obese diabetic/severely combined immunodeficient (NOD/SCID) mice were transplanted with primary CD34⁺ cells or the equivalent starting number of CD34⁺ cells expanded in culture media supplemented by cytokines alone or in combination with 5azaD/TSA, VPA or 5azaD/VPA. As set forth in FIG. 2C, mice transplanted with cells from cultures containing cytokines alone (0 of 5 mice) or cytokines with 5azaD/VPA (0 of 6 mice) were completely devoid of human hematopoietic cell chimerism, demonstrating that these treatments did not expand or preserve pluripotent hematopoietic stem cells. In contrast, all 7 of 7 mice receiving grafts from cultures treated with cytokines and 5azaD/TSA showed evidence of human multi-lineage hematopoietic engraftment (2.6%±0.74%) in recipient mice. Cells from cultures expanded with cytokines and VPA were capable of human hematopoietic engraftment in 2 out of 7 mice with a barely detectable level of chimerism (0.11%, 0.14%) (FIG. 2C). Transplanting equal initial quantities of primary uncultured CD34⁺ cells resulted in human hematopoietic engraftment in 2 of 5 mice. Primary CD34⁺ cells and those expanded in 5azaD/TSA or VPA cultures retained the ability to differentiate into both myeloid and lymphoid lineages following transplantation (FIG. 2D).

### Example 3

### Determination of Severe Combined Immunodeficiency (SCID)

### Repopulating Cell (SRC) Frequency by Limiting Dilution Analyses

The frequency of SCID-repopulating cells (SRCs) present in VPA-expanded, CD34⁺CD90⁺ cord blood-derived stem cells was quantitated in comparison to unmanipulated primary cord blood cells by in vivo xeno-transplant studies using limiting dilution analyses as described previously (Conneally et al., 1997, Proc. Natl. Acad. Sci. 94: 9836-41; Bhatia et al., 1997, J Exp Med. 186: 619-24; Wang et al., 1997, Blood 89: 3919-24; Chute et al., 2005, Blood 105: 576-83). The frequency of SRC was 1 in 22,000 (95% Confidence Interval: 1/11,722-1/41,293) in primary CD34⁺CD90⁺ cells, and 1 in 21,720 (95% Confidence Interval: 1/11,160-1/42,269) in the VPA expanded cultures (Table 5 and FIG. 2E). It was previously demonstrated that cultures containing cytokines alone displayed an SRC frequency of 1 in 123,315, while 5azaD/TSA expanded cultures had an SRC frequency of 1 in 3,147, a 7-fold expansion of the absolute number of SRC in comparison to the input CD34⁺CD90⁺ cells (Araki et al., 2009, Exp Hematol. 37:1084-95). By contrast, VPA-treated cultures prevented SRC loss, and at a minimum maintained SRC numbers during ex-vivo culture despite lacking any detectable bone marrow homing defects.

TABLE 5
Frequency of NOD/SCID repopulating cells in increasing doses of primary
or 5azaD/TSA or VPA treated CD34⁺CD90⁺ cells after 9 days.

| # of primary CD34⁺CD90⁺ | (# of NOD/SCID mice engrafted)/(# of mice transplanted) | | | |
|---|---|---|---|---|
| cells injected on Day 0 or | | Day 9 | Day 9 | Day 9 |
| # of CD34⁺CD90⁺ cells used | | Cytokines | Cytokines | Cytokines |
| to initiate ex vivo cultures | Day 0 | alone | and 5azaD/TSA | and VPA |
| 1,000 | ND | ND | 0/3 | ND |
| 2,000 | 0/6 | ND | 2/4 | 0/7 |
| 5,000 | 3/8 | ND | 4/5 | 2/5 |
| 10,000 | 2/5 | 0/5 | 5/5 | 2/7 |
| 20,000 | 3/5 | 0/3 | 5/5 | 5/8 |
| 50,000 | 4/5 | 2/5 | ND | ND |
| 100,000 | ND | 2/3 | ND | ND |
| SRC Frequency | 1 in 22,000 | 1 in 123,315 | 1 in 3,147 | 1 in 21,720 |
| | (95% CI: 1/11,722- | (95% CI: 1/46,617- | (95% CI: 1/1,602- | (95% CI: 1/11,160- |
| | 1/41,293) | 1/326,200) | 1/6,189) | 1/42,269) |

CI = Confidence Interval, NOD/SCID mice (n = 94) were transplanted with increasing doses of CD34⁺CD90⁺ cells calculated to be present in the purified primary CB CD34⁺ cell fraction or the cellular products of 5azaD/TSA or VPA treated cultures initiated with these numbers of CD34⁺CD90⁺ cells. The data from 7 independent limiting dilution experiments were pooled and analyzed by applying Poisson statistics according to the single-hit model.

In order to assess whether the lower SRC frequency, or lower hematopoietic chimerism, detected following transplantation of VPA expanded cells was due to a defect in the homing of transplanted cells to the host bone marrow, an in vivo homing assay was performed in NOD/SCID mice. The homing efficiency of cord blood cells expanded in control (cytokine-only treated) cultures was 0.05% while 5azaD/TSA- and VPA-expanded CB cells possessed 0.39% and 1.68% homing efficiency, respectively (control vs. 5azaD/TSA, P=0.002; 5azaD/TSA vs. VPA, P=0.008) (FIG. 2F). These data clearly indicated that CMA-expanded CB cells possessed higher bone marrow (BM) homing efficiency than control cells expanded in the absence of CMA. Since VPA-expanded cells possessed higher homing efficiency than 5azaD/TSA expanded cells, their lower SRC frequency and lower chimerism was unlikely to be a result of poor homing efficiency.

### Example 4

### Serial Transplantation Ability of VPA Expanded CB Cells

Long-term function and self-renewal of HSC during ex vivo culture can be demonstrated (albeit indirectly) by hematopoietic reconstitution of secondary hosts after serial transplantation (Hess et al., 2006, Blood. 107: 2162-69). Previously it was shown that 5azaD/TSA-expanded cells are capable of repopulating blood cells in secondary hosts (Araki et al., 2007, Blood 109: 3570-78). Serial transplantation of unfractionated bone marrow from primary recipients engrafted with uncultured (Day 0) CD34⁺ cells resulted in human hematopoietic engraftment in 2 of 5 secondary mice. In contrast, primary recipients engrafted the equivalent input number of CD34⁺CD90⁺ cells expanded with VPA resulted in engraftment of none of the 5 secondary hosts (Table 6). These results indicated that valproic acid can maintain hematopoietic stem cells and promote asymmetric self renewal divisions in culture.

TABLE 6
Comparison between serial transplant capacity of VPA expanded
CB cells in contrast to unmanipulated primary CB cells

| | | ¹Primary transplants | ²Secondary Transplants |
|---|---|---|---|
| | | Primary mouse | Secondary mouse |
| | | BM* chimerism | BM* chimerism |
| Treatment | | (% human) | (% human) |
| 1 | Primary CB | 15.1 | Not Detectable |
| 2 | Primary CB | 29.6 | 10.5 |
| 3 | Primary CB | 16.3 | 0.11 |
| 4 | Primary CB | 48.5 | Not Detectable |
| 5 | Primary CB | 25.3 | Not Detectable |
| 6 | VPA | 0.60 | Not Detectable |
| 7 | VPA | 1.0 | Not Detectable |
| 8 | VPA | 0.30 | Not Detectable |
| 9 | VPA | 5.6 | Not Detectable |
| 10 | VPA | 1.0 | Not Detectable |

¹The progeny of 1 × 10⁴ primary unmanipulated CB were treated ex vivo with VPA for 9 days then injected into sub-lethally irradiated primary mouse.
²10-30 × 10⁶ unseparated BM cells from a primary mouse were injected into sub-lethally irradiated secondary NOD/SCID recipients, which were sacrificed 7 weeks later.
*BM = bone marrow

### Example 5

### The Expression Pattern of Known HSC Self-Renewal Genes in VPA- or 5azaD/TSA-Expanded CD34+ Cells is not Distinct

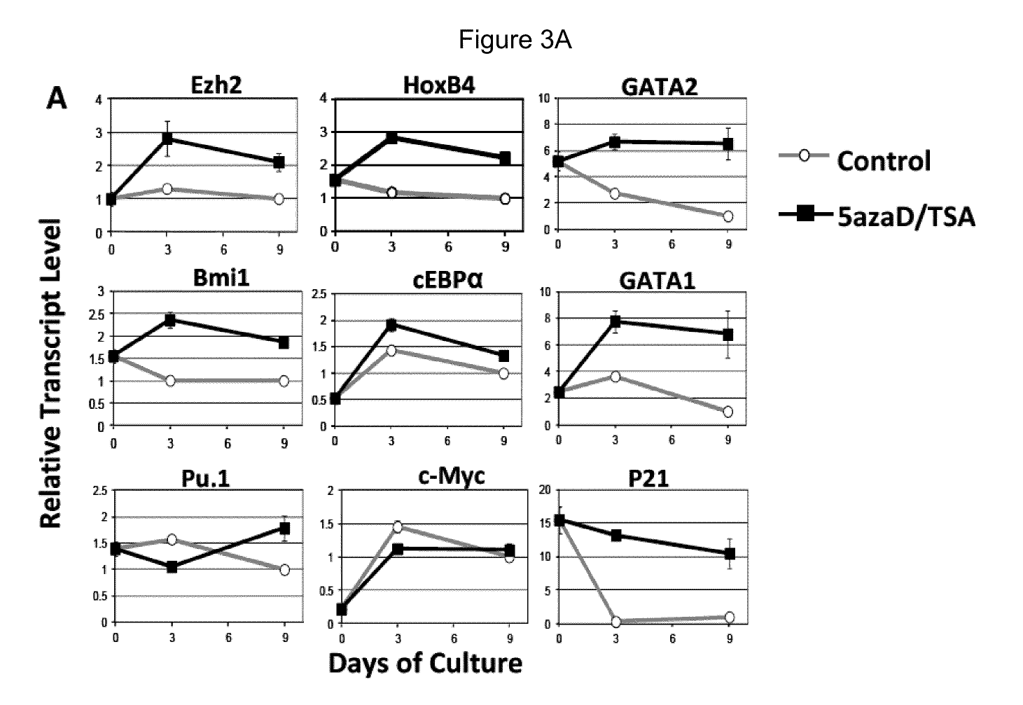

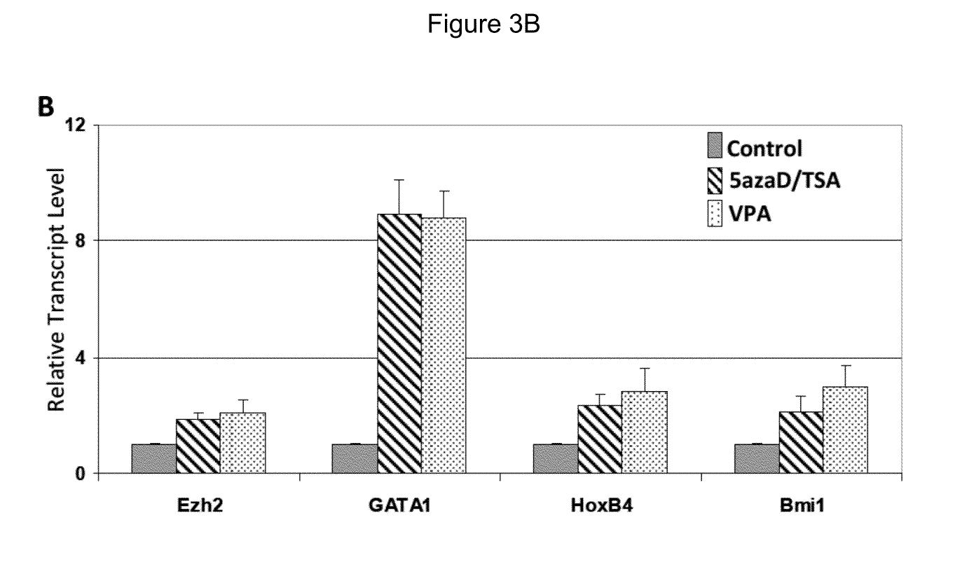

To investigate whether treating cells with CMA altered gene expression, transcript levels of several genes associated with hematopoietic stem cell (HSC) self-renewal and differentiation were compared in unmanipulated primary CD34⁺ cells (day 0) and CD34⁺ cells expanded in the presence or absence of CMA (5azaD/TSA) during ex vivo culture. Since the proportion of primitive CD34⁺ cells expanded in the presence or absence of CMA in the culture was significantly different (presence of 5azaD/TSA, 36.7%±4.4% vs. cytokines alone, 7.0%±0.4%), CD34⁺ cells were re-isolated (>90% purity) following ex vivo expansion in culture and used to perform real time quantitative PCR for comparison between CD34⁺ cells expanded in culture and primary unmanipulated CD34⁺ cells. As shown in FIG. 3A, expression levels of several genes associated with self-renewal, including Ezh2, Bmi1, GATA2, and HoxB4 were maintained for cells cultured with 5azaD/TSA (expression levels tested at Day 3 and Day 9). In the absence of 5azaD/TSA treatment, transcript levels of these genes were reduced over the course of 9 days of culture, correlating well with poor in vivo hematopoietic reconstitution capacity of untreated cells compared to unmanipulated primary or 5azaD/TSA expanded CD34⁺ cells. The transcript levels of GATA1 (p=0.04), GATA2 (p=0.02), HoxB4 (p=0.005) and Bmi1 (p=0.0001) are significantly higher in CD34⁺ cells expanded with 5azaD/TSA in comparison to CD34⁺ cells in control cultures. Differences in the transcript levels of several genes, including cEBPa (p=0.15), c-Myc (p=0.59) and PU.1 (p=0.8) were not statistically significant in 5azaD/TSA-expanded CD34⁺ cells in comparison to control cultures (FIG. 3A). In addition, transcript levels of CDK inhibitor P21, which regulates the G1/S transition of the cell cycle, was reduced in CD34⁺ cells expanded in the absence of CMAs, but was not reduced in 5azaD/TSA expanded cells (p=0.06). Reduced P21 levels are generally associated with shorter G1/S transition and faster rate of cell divisions. This result was consistent with slower cell division rate of the 5azaD/TSA expanded CD34⁺ cells (that display higher levels of P21) compared with CD34⁺ cells from control cultures. This was also consistent with previous results showing membrane tracking dye as well as bromodeoxyuridine (BrdU) pulse chase assays of CD34⁺CD90⁺ cells during expansion culture (Araki et al., 2007, Blood 109: 3570-78). As shown in FIG. 3B, there was no significant difference between expression of genes generally implicated in HSC self-renewal (including Ezh2, HoxB4, and Bmi1) in 5azaD/TSA and VPA-expanded CD34⁺ cells. Transcript levels of GATA1 were also significantly increased in both 5azaD/TSA and VPA-expanded CD34⁺ cells when compared with control cultures. These results indicated that genes commonly implicated in hematopoietic stem cell maintenance are involved both in maintenance and expansion.

### Example 6

### Identification of Distinct HSC Expansion and Maintenance Gene Sets

Analyses of global gene expression between CD34⁺ cells expanded with 5azaD/TSA or VPA and control cultures were performed, and distinct gene expression profiles in expanded CD34⁺ cells were found in cells linked with in vivo hematopoietic repopulation function. These gene expression profiles represent: expansion (5azaD/TSA), maintenance (VPA) or loss (control). This profile was ascertained by performing global gene expression array that identified genes differentially expressed between CD34⁺ cells expanded with 5azaD/TSA or control cultures. The differential expression pattern revealed from CD34⁺ cells expanded in 5azaD/TSA (expansion) genes vs. CD34⁺ cells expanded in control cultures were associated with both HSC expansion and maintenance (Group A), while those differentially expressed between 5azaD/TSA vs. VPA expanded CD34⁺ cells (Group B) were involved in HSC expansion but not maintenance.

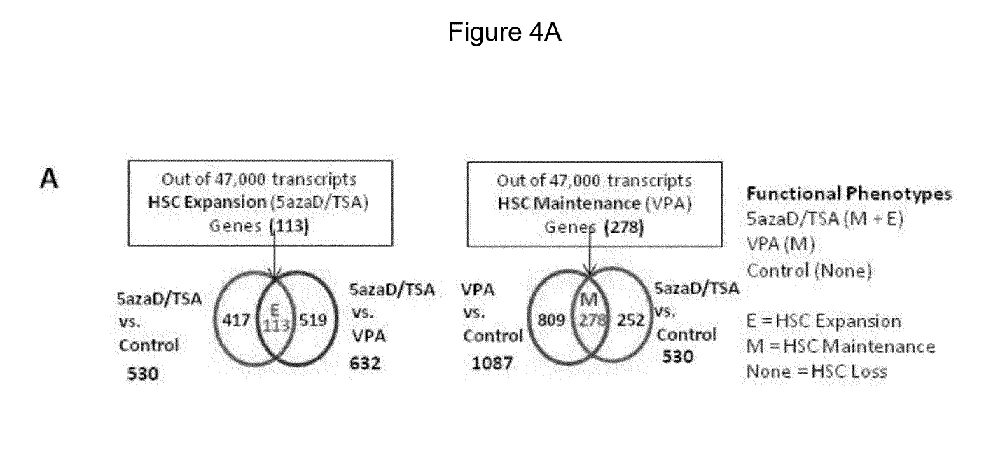

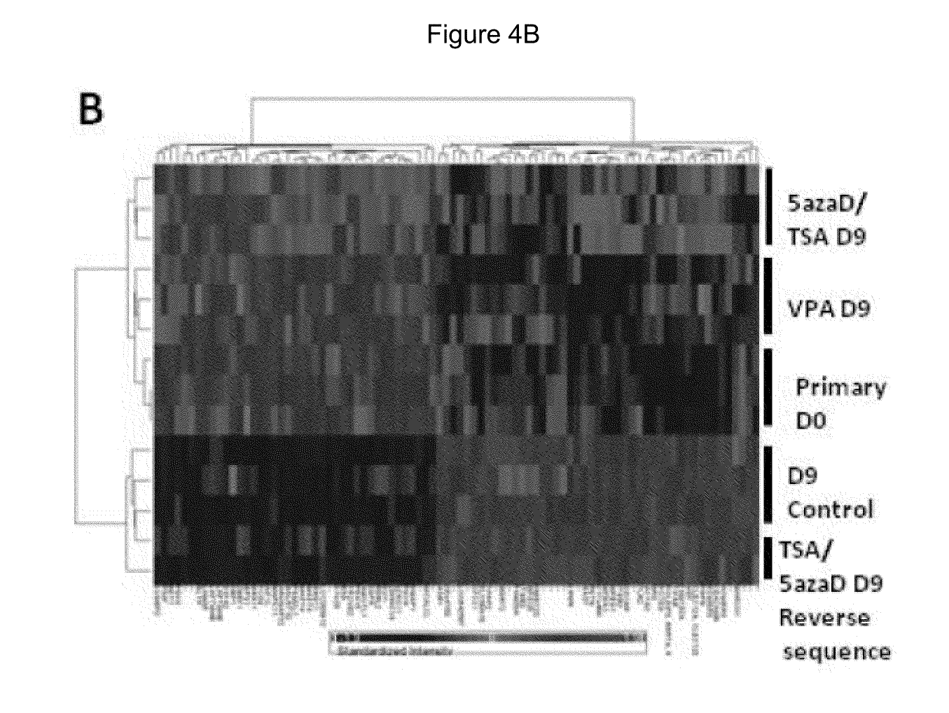

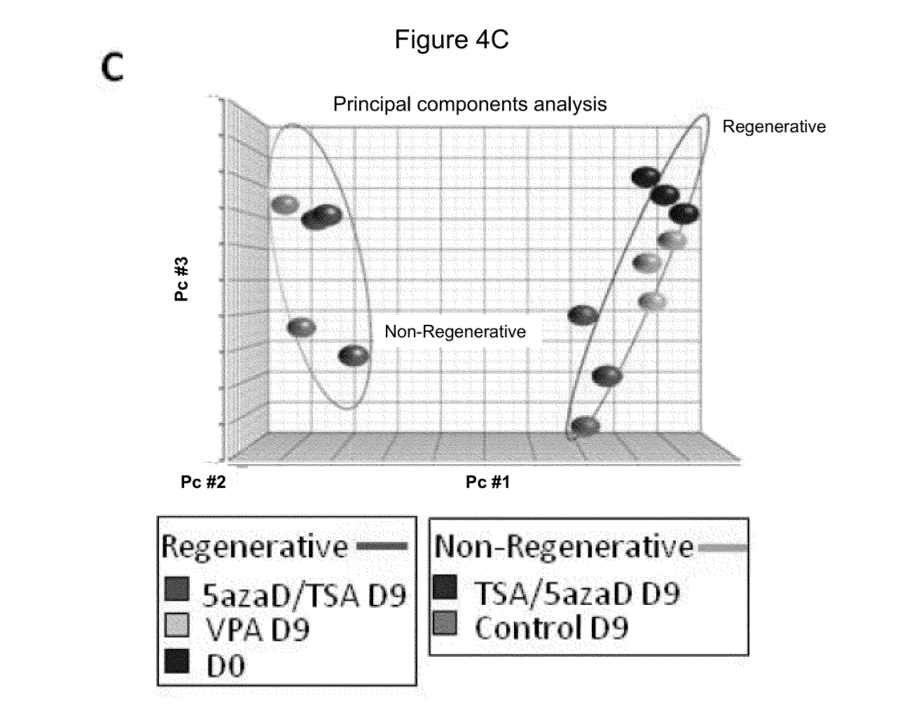

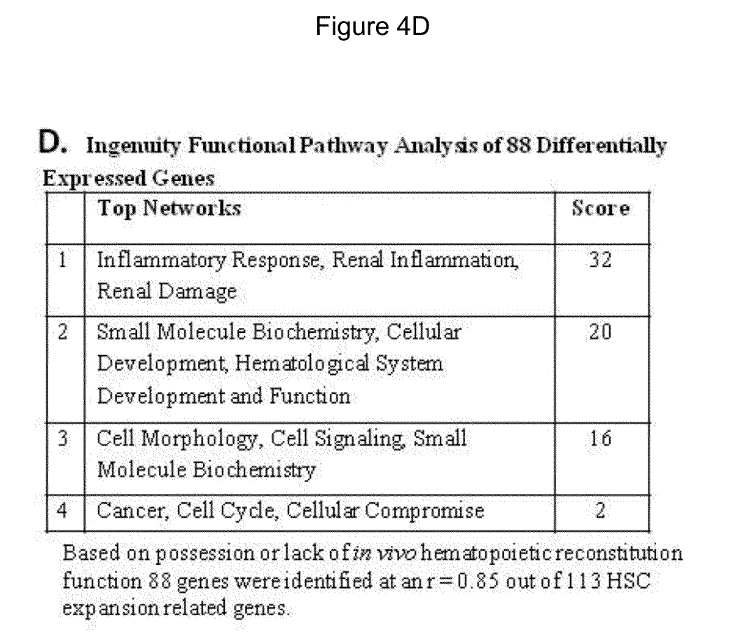

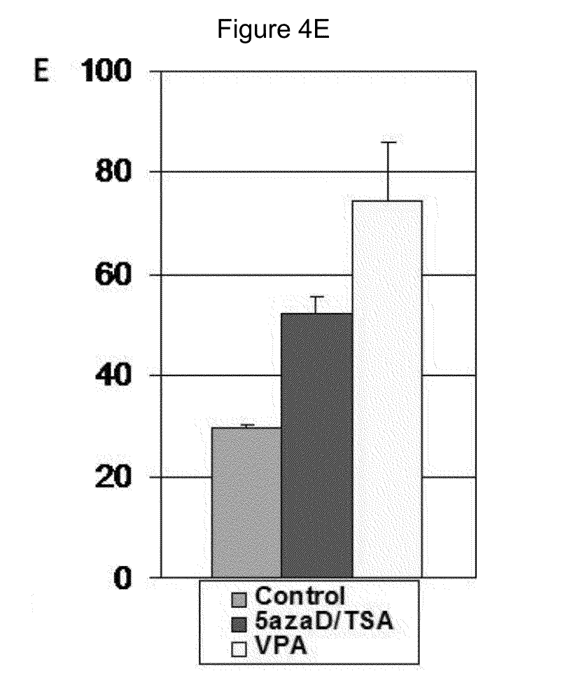

The genes shared between Groups A (5azaD/TSA vs. Control) and B (5azaD/TSA vs. VPA) expanded CD34⁺ cells were exclusively associated with HSC expansion (A vs. B). By determining the intersections of these gene sets, a list of 113 common genes were found to be differentially expressed between 5azaD/TSA vs. control and 5azaD/TSA vs. VPA expanded CD34⁺ cells which are functionally linked with HSC expansion (5azaD/TSA), as demonstrated by in vivo hematopoietic reconstitution assays. Similarly, 278 common genes that are functionally related to HSC maintenance (VPA) were identified to be differentially expressed between 5azaD/TSA vs. control and VPA vs. control derived CD34⁺ cells (FIG. 4A) Intriguingly, Ingenuity® Functional Pathway Analysis linked the 113 HSC expansion genes with molecules involved in inflammation and lipid metabolism which are distinct from 278 HSC maintenance genes related pathways (Tables 7-10).

TABLE 7
Ingenuity Pathway Analyses: 113 HSC Expansion
Genes: Associated Network Functions

| | Top Networks | Score |
|---|---|---|
| 1 | Cellular Movement, Cellular Growth and | 47 |
| | Proliferation, Cancer | |
| 2 | Cellular Development, Cellular Growth and | 32 |
| | Proliferation, Cell Cycle | |
| 3 | Cell Cycle, Nervous System Development and | 26 |
| | Function, Lipid Metabolism | |
| 4 | Inflammatory Response, Cellular Movement, | 24 |
| | Immune Cell Trafficking | |

Global functional analysis conducted using Ingenuity ® Pathway Analysis

TABLE 8
Ingenuity Pathway Analyses: 113 HSC Expansion
Related Genes: Molecular and Cellular Functions

| Name | p-value | # Molecules |
|---|---|---|
| Cellular Movement | 1.06E−05-1.92E−02 | 28 |
| Lipid Metabolism | 4.12E−05-1.92E−02 | 14 |
| Molecular Transport | 4.12E−05-1.92E−02 | 12 |
| Small Molecule | 4.12E−05-1.92E−02 | 22 |
| Biochemistry | | |
| Cell-to-Cell signaling | 1.23E−04-1.92E−02 | 20 |
| and interaction | | |

TABLE 9
Functional Pathway Analysis of 278 HSC Maintenance Genes: Associated
Network Functions

| | Top Networks | Score |
|---|---|---|
| 1 | Cellular Function and Maintenance, Molecular Transport, | 48 |
| | Gene Expression | |
| 2 | Cellular Movement, Cell Morphology, Cellular Growth and | 38 |
| | Proliferation | |
| 3 | Cellular Growth and Proliferation, Cell Cycle, DNA | 37 |
| | Replication, Recombination, and Repair | |
| 4 | Cellular Development, Hematopoiesis, Gene Expression | 28 |

TABLE 10
278 HSC Maintenance Related Genes:
Molecular and Cellular Functions

| Name | p-value | # Molecules |
|---|---|---|
| Cellular Development | 1.47E−09-3.89-03 | 74 |
| Cellular Growth and | 1.87E−08-4.16E−03 | 80 |
| Proliferation | | |
| Cellular Movement | 4.34E−08-4.48E−03 | 57 |
| Cell Death | 3.47E−06-4.57E−03 | 66 |
| Gene Expression | 5.93E−06-4.89E−03 | 52 |

In addition, differential gene expression of primary or ex vivo expanded CD34⁺ cells was analyzed using a heat map based on their in vivo hematopoietic reconstitution function. Primary unmanipulated or 5azaD/TSA or VPA expanded CD34⁺ cell populations possessing regenerative potential (Day 0, 5azaD/TSA, and VPA) were assigned a capacity grade of 2, while non-regenerative samples (Day 9 control and Day 9 TSA/5azaD) were assigned a grade of 0.01. As shown in the heat map in FIG. 4B, 88 genes were found to have an r-value of 0.85, indicating a high level of correlation between the expression pattern of these genes and the regenerative capacity of the samples. Furthermore, Principal Component Analysis of these 88 genes also revealed that samples with regenerative capacity (Day 0, 5azaD/TSA, and VPA) are clustered together, while samples without regenerative capacity (Day 9 control and D9 TSA/5azaD) were clustered separately in a distinct region, indicating the potential for unique gene function (FIG. 4C). In addition, Ingenuity® Functional Pathway Analysis of these 88 genes detected inflammation as one of the top networks with the highest score (FIG. 4D). In support of involvement of signals involving inflammation, an ELISA assay revealed that the level of inflammatory mediator leukotriene B4 (LTB4) in conditioned medium from expansion of CD34+ cells with 5azaD/TSA and VPA was increased compared to control (FIG. 4E). The presence of an increase in inflammatory mediators such as, LTB4 in the cultures is consistent with at least a partial role for these molecules in hematopoietic stem or progenitor cell expansion and maintenance. Of interest, LTB4 is synthesized from arachidonic acid by dual action of Alox5 gene product, 5-lipooxygenase and LTA4 hydrolase, further supporting involvement of signals involving inflammation/lipid metabolism potentially governing hematopoietic stem cell fate choices in culture.

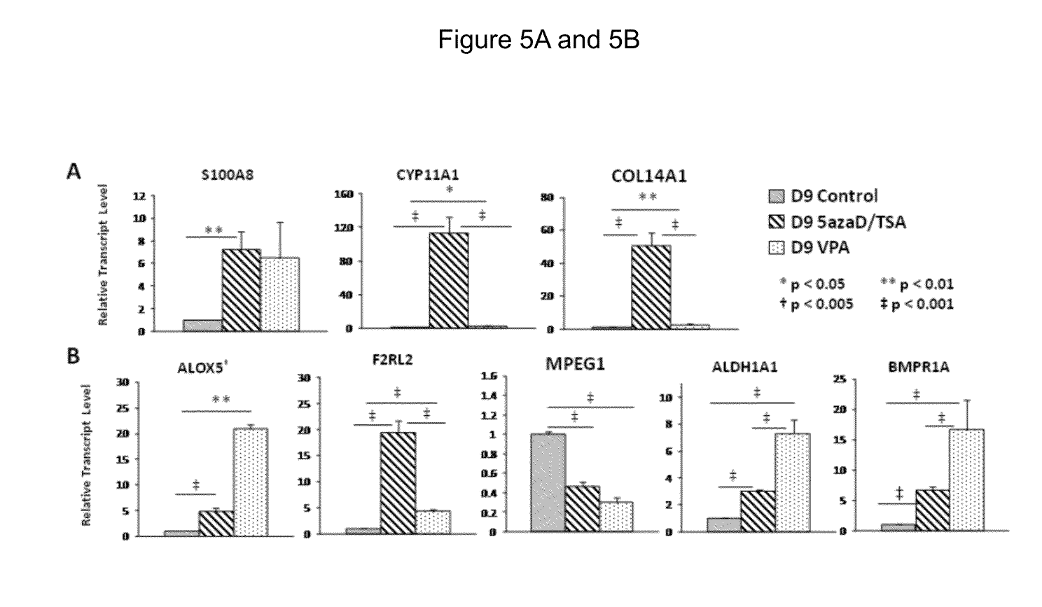

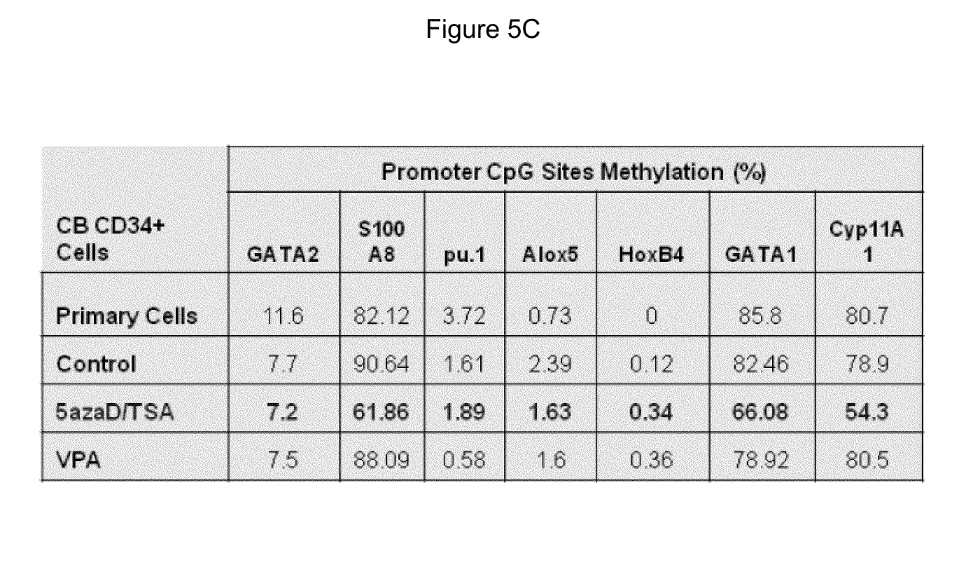

Transcript levels of several genes selected from the list of 113 HSC expansion or 278 HSC maintenance genes were verified using real time qPCR (FIG. 5A, FIG. 5B; using primers set forth in Table 1). It was notable that there were 19 shared genes between the list of 113 HSC expansion and 278 HSC maintenance related genes which included genes involved in lipid metabolism, such as F2RL2, MPEG1, ALDH1A1, and BMPR1A (FIG. 5A). These shared genes likely possess dual functions: HSC maintenance and expansion. Thrombin receptor F2RL2 gene transcript was represented in both HSC expansion and HSC maintenance gene list. However, the PCR validation data indicated that F2RL2 was differentially expressed in CD34⁺ cells expanded in 5azaD/TSA in contrast to VPA (FIG. 5B). Similarly, Cyp11A1 was not in the HSC maintenance group, which was consistent with PCR validation as demonstrated by lower transcript levels in VPA expanded CD34⁺ cells than 5azaD/TSA (FIG. 5A). The differentially expressed genes representative of the HSC expansion list included calcium binding protein S100A8 and the HSC maintenance list included Alox5, a gene involved in arachidonic acid metabolism and production of leukotrienes. Both genes function as inflammatory mediators and their transcript levels were increased in 5azaD/TSA as well as in VPA-expanded CD34⁺ cells following culture in contrast to controls (FIGS. 5A, 5B). Interestingly, the transcript levels of Alox5 were higher in CD34⁺ cells expanded in VPA (FIG. 5B) in contrast to 5azaD/TSA. The higher expression of Alox5 in VPA-expanded CD34⁺ cells was also consistent with the microarray results, as Alox5 was one of the 278 HSC maintenance related genes. Similarly, transcript levels of maintenance genes, including Alox5, ALDH1A1 and BMPR1A, were relatively higher in CD34+ cells expanded in VPA than in 5azaD/TSA (FIG. 5B).

### Example 7

### Epigenetics Likely Exerts Both Direct and Indirect Effects to Promote HSC Expansion

Experiments were performed to quantify gene-specific and global methylation of genomic DNA by pyrosequencing. Genes implicated in HSC self-renewal, including HoxB4 and GATA2, almost completely lacked methylation despite the fact that their transcript levels were increased following CMA treatment in culture (FIG. 5C). Genes known for their role in hematopoiesis, including GATA1, were found to be methylated in uncultured CD34⁺ cells and control cultures, while 5azaD/TSA treatment resulted in significant demethylation corresponding with their relatively higher transcript levels (FIG. 5C). Although VPA treatment resulted in minimal demethylation of the GATA1 gene after culture, increased GATA1 transcript levels were also observed in this condition (albeit likely to be an indirect effect (see FIG. 3B). CD34⁺ cells cultured in the absence of CMAs had methylation levels similar to uncultured primary CD34⁺ cells for all genes tested (FIG. 5C).

Transcript levels of several genes not known for their role in hematopoiesis were not only increased following CMA treatment during ex vivo culture, but also were found to be exclusively associated with HSC expansion (113 genes, e.g. S100A8). Consistently, 5azaD/TSA-expanded CD34⁺ cells displayed significant reduction in the methylation levels of 5100A8 and Cyp11A1 gene promoter sites (FIG. 5C). Cyp11A1 gene transcript levels were also significantly higher in 5azaD/TSA-expanded CD34⁺ cells in contrast to CD34⁺ cells expanded in VPA or control cultures (5azaD/TSA vs. Control p=<0.0001, 5azaD/TSA vs. VPA p=<0.0001). VPA-expanded CD34⁺ cells almost completely lacked any changes in methylation despite increase in their transcript levels of S100A8. Similarly, although the transcript levels of Alox5 in 5azaD/TSA-expanded CD34⁺ cells was found to be increased, it lacked any detectable methylation suggesting that Alox5 gene transcript levels may be regulated by some other indirect mechanisms.

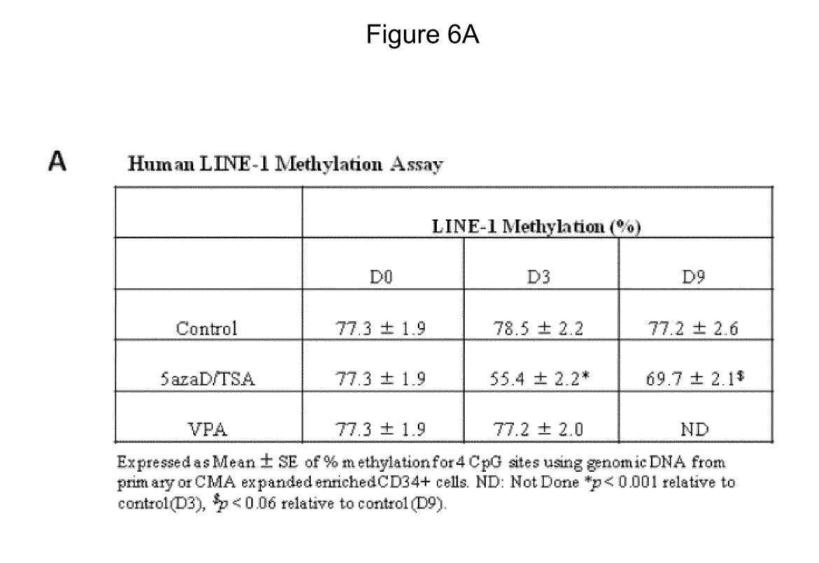

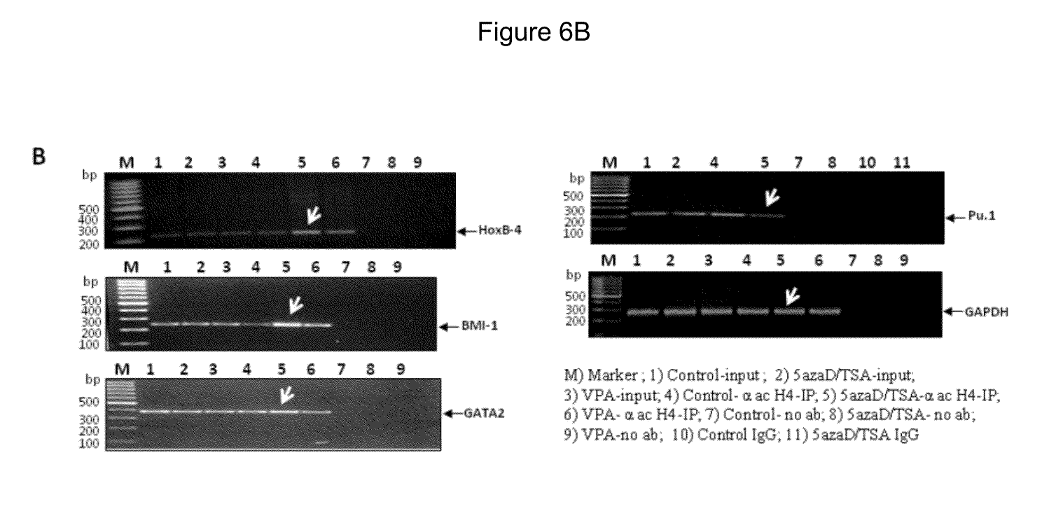

Global methylation of genomic DNA was quantified by assaying long interspersed nucleotide element 1 (LINE 1) methylation as a surrogate marker (Yang et al., 2004, Nucleic Acids Res. 32: e38). The degree of global methylation of CD34⁺ cells prior to and following ex vivo culture in the presence or absence of CMAs (5azaD/TSA and VPA) was determined in these experiments. Four CpG sites within LINE 1 elements were analyzed at day 0 and the mean methylation for unmanipulated primary CD34⁺ cells was found to be 77.3%±1.9%, while at day 3, CD34⁺ cells cultured in the absence of CMA, had 78.5%±2.2% methylation. CD34⁺ cells cultured in 5azaD/TSA and VPA had 55.4%±2.2% and 77.2%±2.0% methylation respectively at day 3 (FIG. 6A). These results clearly indicated that culturing CD34⁺ cells in 5azaD/TSA resulted in significant hypomethylation in these cells, compared with unmanipulated CD34⁺ cells, CD34⁺ cells expanded in the presence of VPA in the culture media or control cultures. CD34⁺ cells cultured in the absence of 5azaD/TSA treatment showed that methylation levels of CpG sites in LINEs remained at a high level.

### Example 8

### Methylation of Gene Promoter Sites and HSC Expansion

In order to determine the relationship between CD34⁺ cell exposure to CMA and alteration of gene transcript levels, the degree of CpG methylation at promoter sites of genes selected from the list of 113 genes exclusively linked with transplantable HSC expansion function were quantified. Genes involved in promoting HSC expansion were silenced by epigenetic mechanisms during ex vivo expansion in control cultures, and reactivation of these genes by addition of CMAs in culture media promoted expansion of transplantable HSC.

The validated genes included Group A (representing 113 HSC expansion related genes) and Group B (278 HSC maintenance related genes) as described above. There were some shared genes between these two functional groups of genes identified from global microarray (47,000 transcripts), including: F2RL2, MPEGI, ALDH1A1, BMPR1A, calcium binding protein gene S100A8 and Alox5, a gene involved in arachidonic acid metabolism and production of leukotrienes (the products of the latter two genes function as inflammatory mediators).

While transcript levels of both S100A8 and Alox5 (genes linked to inflammatory pathways) were higher in 5azaD/TSA- and VPA-expanded CD34⁺ cells, transcript levels of Alox5 were significantly higher in CD34⁺ cells expanded in VPA in contrast to CD34⁺ cells expanded 5azaD/TSA (thus Alox5 levels were associated with distinct outcomes, relatively lower Alox5 levels following 5azaD/TSA treatment led to expansion, while higher Alox5 levels following VPA treatment led to maintenance of transplantable HSCs). The results showing higher expression of Alox5 in VPA-expanded CD34⁺ cells was also consistent with microarray assay and ELISA assay results. In contrast, other genes tested (including HoxB4, GATA2, Ezh2, and PU.1) showed relatively higher expression in both 5azaD/TSA- and VPA-expanded CD34⁺ cells compared with control cultures.

Gene transcript levels determined by quantitative RT-PCR did not permit discrimination between CD34⁺ cells expanded in 5azaD/TSA or VPA, but differential microarray assay for genes enriched based on their in vivo function potential identified HSC expansion and maintenance related genes, many of which had differential gene expression patterns. For instance, the expression of genes including Cyp11A1, Col14 A1, F2RL2 were relatively higher in CD34⁺ cells expanded in 5azaD/TSA than CD34⁺ cells expanded in VPA (FIGS. 5A and 5B). Similarly, transcript levels of genes including Alox5, ALDH1A1 and BMPR1A was relatively higher in VPA expanded CD34⁺ cells than CD34⁺ cells expanded in 5azaD/TSA. HoxB4, a gene implicated in HSC self-renewal, almost completely lacked any detectable methylation by promoter site CpG gyro-sequencing (FIG. 5C). Genes known for their role in hematopoiesis, including GATA1, were methylated in primary unmanipulated CD34⁺ cells and control cultures while 5azaD/TSA treatment resulted in significant demethylation consistent with their higher transcript levels. Although VPA resulted in minimal demethylation of the GATA1 gene after culture, increased GATA1 transcript levels were also observed (FIG. 3B). CD34⁺ cells cultured in the absence of CMAs had methylation levels similar to unmanipulated primary CD34⁺ cells (FIG. 5C). The relatively higher methylation of the GATA1 promoter observed in CD34⁺ cells from control cultures and lower methylation of 5azaD/TSA treated expanded CD34⁺ cells corresponded with their relative GATA1 transcript levels as assayed by PCR (FIG. 3B). Furthermore, transcript levels of several genes not known for their role in hematopoiesis were not only increased following CMA treatment during ex vivo culture, but also were found to be exclusively associated with 5azaD/TSA expanded CD34⁺ cells (HSC expansion), but not in VPA expanded (HSC Maintenance) or CD34⁺ cells from control cultures (HSC loss) by differential gene expression analyses. Sequencing of bisulfate-modified DNA showed relative hypomethylation of CpG sites of the S100A8 gene promoter in 5azaD/TSA expanded CD34⁺ cells in comparison to control or unmanipulated primary CD34⁺ cells, and that VPA expanded cells almost completely lacked any changes in methylation (FIG. 5C). However despite increased transcript levels of Alox5 in 5azaD/TSA-expanded CD34⁺ cells, CpG sites from Alox5 gene promoter lacked significant change in CD34⁺ cells in all culture conditions (FIG. 5C), suggesting that Alox5 gene regulation may be due to indirect effects of CMA treatment or methylation changes involving other sites in DNA (Ji et al., 2010, Nature 467: 338-342).

### Example 9

### 5azaD/TSA and VPA can Alter Histone Acetylation of Gene Promoter Sites

In order to examine whether the alteration of gene expression observed in CD34⁺ cells during ex vivo expansion culture with CMA was due to changes in histone acetylation, acetylation of histone H4 was analyzed for several gene promoter sites including HoxB4, Bmi1, GATA2, PU.1, as well as GAPDH as a control. Standard chromatin immunoprecipitation (ChIP) assays were used employing an antibody specific to acetylated histone H4 as described previously (Sankar et al., 2008, Oncogene 27:5717-28). Increased histone H4 acetylation of the promoter regions of HoxB4, Bmi1, and GATA2 genes were found in both 5azaD/TSA and VPA expanded CD34⁺ cells in contrast to control cultures, corresponding to their higher transcript levels. More promoter fragments for Bmi1, HoxB4, and GATA2, and less fragments for PU.1 were amplified in 5azaD/TSA expanded CD34⁺ cells as compared to control and VPA-expanded CD34⁺ cells (FIG. 6B). Notably, VPA-expanded CD34⁺ cells showed intermediate levels of histone H4 acetylation for Bmi1 and HoxB4 genes compared to control cultures (FIG. 6B). Together, these data supported the conclusion that 5azaD/TSA- and VPA-expanded CD34⁺ cells increased histone H4 acetylation at the promoter sites of genes whose transcription level was positively correlated with their degree of acetylation.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

We claim:

1. A method for preparing an expanded population of hematopoietic progenitor cells from a biological source comprising said cells, comprising the step of culturing at least a portion of a cell preparation in a culture media containing valproic acid for a time and at a concentration wherein the population of hematopoietic progenitor cells is expanded and wherein hematopoietic stem cells in said cell preparation are maintained.

2. The method of claim 1 wherein the biological source is umbilical cord blood, growth factor mobilized peripheral blood cells, or bone marrow cells.

3. The method of claim 1, wherein hematopoietic progenitor cells are grown in culture for between about 7 to about 9 days in which valproic acid is added in culture at least twice once at 0 hr at the start and after 48 hrs of culture.

4. The method of claim 3, wherein the hematopoietic progenitor cells are grown in culture media containing valproic acid between about 7 and 9 days.

5. The method of claim 1, wherein valproic acid is in the culture media at a concentration of about 0.5 to 1 mM

6. The method of claim 1, wherein said medium comprises at least one cytokine selected from the group consisting of stem cell factor (SCF), Interleukin-1 (IL-1), Interleukin-2 (IL-2), Interleukin-3 (IL-3), Interleukin-4 (IL-4), Interleukin-5 (IL-5), Interleukin-6 (IL-6), Interleukin-7 (IL-7), Interleukin-8 (IL-8), Interleukin-9 (IL-9), Interleukin-10 (IL-10), Interleukin-11 (IL-11), Interleukin-12 (IL-12), erythropoietin (EPO), thrombopoietin (TPO), Granulocyte Colony-stimulating Growth Factor (G-CSF), Macrophage Colony-Stimulating Factor (M-CSF), Granulocyte-Macrophage Colony-Stimulating Factor (GM-CSF), Insulin-like Growth Factor-1 (IGF-1), Flt-3 ligand, and Leukemic Inhibitory Factor (LIF).

7. The method of claim 6, wherein the media comprises Flt-3 ligand, TPO, IL-3 and SCF for the first 48 hours followed by replacement of fresh media supplemented with Flt-3 ligand, TPO and SCF.

8. A hematopoietic progenitor cell preparation prepared according to the method of claim 1.

9. A pharmaceutical composition comprising a hematopoietic progenitor cell population according to claim 8 and a pharmaceutically acceptable carrier or adjuvant.

10. A method for preventing or treating a cancer chemotherapy patient for hematopoietic sequellae of cancer chemotherapy, comprising the step of administering to the patient a pharmaceutical composition of hematopoietic progenitor cells of claim 8.

11. The method of claim 10, wherein the hematopoietic sequella is neutropenia.

12. The method of claim 10, wherein the hematopoietic sequella is leukocytopenia, pancytopenia or thrombocytopenia.

13. A method for preparing a composite cell preparation from a biological source comprising hematopoietic stem cells and hematopoietic progenitor cells, comprising the step of culturing a portion of said umbilical cord cell preparation in the presence of valproic acid for a time and at a concentration wherein the population of hematopoietic progenitor cells is expanded, and combining said expanded portion with an unmanipulated portion of umbilical cord blood into the composite cell preparation.

14. A method for preparing a composite cell preparation from a biological source comprising hematopoietic stem cells and hematopoietic progenitor cells, comprising the step of culturing a portion of said umbilical cord cell preparation in the presence of valproic acid for a time and at a concentration wherein the population of hematopoietic progenitor cells is expanded, and culturing a second portion of said umbilical cord blood stem cell preparation sequentially in the presence of 5-aza-2′-deoxycytidine and trichostatin A for a time and at a concentration wherein the population of hematopoietic stem cells is expanded, and combining said first and second expanded portions of hematopoietic stem cells and hematopoietic progenitor cells into the composite cell preparation.

15. The method of claim 13 wherein the biological source is umbilical cord blood, growth factor mobilized peripheral blood cells, or bone marrow cells.

16. A composite cell preparation comprising an expanded population of hematopoietic progenitor cells and unmanipulated umbilical cord blood cells prepared according to claim 13.

17. A composite cell preparation comprising an expanded population of hematopoietic progenitor cells and hematopoietic stem cells prepared according to claim 14.

18. A pharmaceutical composition comprising a composite cell preparation according to claim 16 and a pharmaceutically acceptable carrier or adjuvant.

19. A pharmaceutical composition comprising a composite cell preparation according to claim 17 and a pharmaceutically acceptable carrier or adjuvant.

20. A method for repopulating bone marrow in an animal with hematopoietic stem cells or hematopoietic progenitor cells or both, comprising the step of administering to an animal in need thereof a pharmaceutical composition according to claim 18.

21. A method according to claim 14, wherein said first portion comprises about one third of the composition and said second portion comprises about two thirds of the composition.

22. The method of claims 13, wherein hematopoietic progenitor cells are grown in culture for between about 2 to about 9 days prior to the addition of valproic acid.

23. The method of claim 22, wherein the hematopoietic progenitor cells are grown in culture media containing valproic acid between about 7 and 9 days.

24. The method of claims 13, wherein valproic acid is in the culture media at a concentration of about 0.5 to 1.0 mM.

25. The method of claims 13, wherein said medium comprises at least one cytokine selected from the group consisting of stem cell factor (SCF), Interleukin-1 (IL-1), Interleukin-2 (IL-2), Interleukin-3 (IL-3), Interleukin-4 (IL-4), Interleukin-5 (IL-5), Interleukin-6 (IL-6), Interleukin-7 (IL-7), Interleukin-8 (IL-8), Interleukin-9 (IL-9), Interleukin-10 (IL-10), Interleukin-11 (IL-11), Interleukin-12 (IL-12), erythropoietin (EPO), thrombopoietin (TPO), Granulocyte Colony-stimulating Growth Factor (G-CSF), Macrophage Colony-Stimulating Factor (M-CSF), Granulocyte-Macrophage Colony-Stimulating Factor (GM-CSF), Insulin-like Growth Factor-1 (IGF-1), Flt-3 ligand, and Leukemic Inhibitory Factor (LIF).

26. The method of claim 25, wherein the media comprises Flt-3 ligand, TPO, IL-3 and SCF for the first 48 hours followed by fresh medium comprised of Flt-3 ligand, TPO, and SCF.

27. A method for assessing functional potency of a blood cell preparation comprising hematopoietic progenitor cells and hematopoietic stem cells from a biological source for a capacity to prevent or treat hematopoietic sequellae of cancer chemotherapy, the method comprising the steps of monitoring expression of genes Alox5 or F2RL2 as biomarkers in an expanded hematopoietic progenitor cell culture or an expanded hematopoietic stem cell culture from said biological source, wherein said hematopoietic progenitor cells are expanded in the presence of valproic acid and said hematopoietic stem cells are expanded in the presence sequentially of 5-aza-2′-deoxycytidine and trichostatin A, wherein detecting expression of Alox5 or F2RL2, S100A8, Cyp11A1 or Collagen 14A1 genes that is greater than expression of said genes in unmanipulated cell population indicates that the cell preparation has a capacity for treating or preventing hematopoietic sequellae of cancer chemotherapy, radiation therapy or exposure to high dose radiation comprising complete ablation of bone marrow blood producing capacity.

28. A method for assessing a blood cell preparation from a biological source comprising hematopoietic progenitor cells for a capacity to repopulate hematopoietic progenitor cells in a patient in need thereof, the method comprising the steps of assaying said cell preparation for expression of Alox5 or F2RL2, S100A8, Cyp11A1, or Collagen14A1, wherein detecting expression of Alox5 or F2RL2, S100A8, Cyp11A1 or Collagen 14A1 genes that is greater than expression of said genes in unmanipulated cell population indicates that the preparation has a capacity for repopulate repopulate hematopoietic progenitor cells in said patient.

29. The method of claims 27, wherein the biological source is umbilical cord blood, growth factor mobilized peripheral blood cells, or bone marrow cells.

30. The method of claim 14 wherein the biological source is umbilical cord blood, growth factor mobilized peripheral blood cells, or bone marrow cells.

31. A method for repopulating bone marrow in an animal with hematopoietic stem cells or hematopoietic progenitor cells or both, comprising the step of administering to an animal in need thereof a pharmaceutical composition according to claim 19.

32. The method of claims 14, wherein hematopoietic progenitor cells are grown in culture for between about 2 to about 9 days prior to the addition of valproic acid.

33. The method of claim 32, wherein the hematopoietic progenitor cells are grown in culture media containing valproic acid between about 7 and 9 days.

34. The method of claims 14, wherein valproic acid is in the culture media at a concentration of about 0.5 to 1.0 mM.

35. The method of claims 14, wherein said medium comprises at least one cytokine selected from the group consisting of stem cell factor (SCF), Interleukin-1 (IL-1), Interleukin-2 (IL-2), Interleukin-3 (IL-3), Interleukin-4 (IL-4), Interleukin-5 (IL-5), Interleukin-6 (IL-6), Interleukin-7 (IL-7), Interleukin-8 (IL-8), Interleukin-9 (IL-9), Interleukin-10 (IL-10), Interleukin-11 (IL-11), Interleukin-12 (IL-12), erythropoietin (EPO), thrombopoietin (TPO), Granulocyte Colony-stimulating Growth Factor (G-CSF), Macrophage Colony-Stimulating Factor (M-CSF), Granulocyte-Macrophage Colony-Stimulating Factor (GM-CSF), Insulin-like Growth Factor-1 (IGF-1), Flt-3 ligand, and Leukemic Inhibitory Factor (LIF).

36. The method of claim 35, wherein the media comprises Flt-3 ligand, TPO, IL-3 and SCF for the first 48 hours followed by fresh medium comprised of Flt-3 ligand, TPO, and SCF.

37. The method of claims 28, wherein the biological source is umbilical cord blood, growth factor mobilized peripheral blood cells, or bone marrow cells.
